(19) United States
(12) Patent Application Publication
(10) Pub. No.: US 2025/0161369 A1
(43) Pub. Date: May 22, 2025

(54) Compositions and Methods to Modulate the Immune System

(71) Applicant: Aloe Therapeutics Inc, Darnestown, MD (US)

(72) Inventor: Martha SKLAVOS, Darnestown, MD (US)

(21) Appl. No.: 18/810,587

(22) Filed: Aug. 21, 2024

Related U.S. Application Data
(63) Continuation of application No. PCT/US2023/062984, filed on Feb. 22, 2023.
(60) Provisional application No. 63/312,947, filed on Feb. 23, 2022.

(51) Int. Cl. A61K 35/35; A61K 38/17; A61P 35/00

(52) U.S. Cl. CPC A61K 35/35; A61K 38/1774; A61P 35/00

(57) ABSTRACT

Provided herein are compositions and methods for inducing an immune response, treating a disease or condition, or a combination thereof. The compositions and methods as described herein may provide a cancer treatment. The compositions and methods as described herein may provide enhanced efficacy of a cancer therapy, such as a checkpoint inhibitor treatment. The compositions and methods as described herein may provide an altered immune response, such as in or proximal to a cancer or tumor microenvironment.

### CROSS-REFERENCE

This application is a continuation application of PCT Application No. PCT/US2023/062984, filed Feb. 22, 2023, which claims the benefit of U.S. Provisional Application No. 63/312,947, filed Feb. 23, 2022, the disclosures of which are incorporated herein by reference in their entirety.

### SUMMARY OF THE INVENTION

Disclosed herein are methods of treating a subject having a cancer. In some embodiments, the method can comprise: administering a therapeutically effective amount of an allogeneic cell and/or a xenogeneic cell to the subject, wherein an antigen marker profile of a cell of the subject comprises at least one mismatched antigen marker as compared to an antigen marker profile of the allogeneic cell or the xenogeneic cell administered to the subject. In some embodiments, a cancer can comprise a malignant tumor. In some embodiments, an antigen marker profile can comprise a human leukocyte antigen (HLA) or a portion thereof. In some embodiments, at least one mismatched antigen marker can comprise 2 mismatched antigen markers. In some embodiments, at least one mismatched antigen marker can comprise 3 mismatched antigen markers. In some embodiments, a cell transplant or tissue transplant comprising the allogeneic cell or the xenogeneic cell can be administered to the subject. In some embodiments, a method can further comprise: administering an isolated and purified HLA or a portion thereof to the subject, wherein the isolated and purified HLA or the portion thereof can be mismatched to an antigen marker profile of a cell of the subject. In some embodiments, an HLA or portion thereof can be a recombinant HLA or recombinant portion thereof. In some embodiments, a method can further comprise administering a cancer therapy to the subject. In some embodiments, an allogeneic cell or a xenogeneic cell and a cancer therapy can be administered concurrently. In some embodiments, an allogeneic cell or a xenogeneic cell and a cancer therapy can be administered consecutively. In some embodiments, a cancer therapy can comprise at least two cancer therapies. In some embodiments, a cancer therapy can comprise a chimeric antigen receptor (CAR) T cell therapy, an immunotherapy, a chemotherapy, a radiation therapy, a surgery, a hormone therapy, a bone marrow transplant, a small molecule, a biologic, or any combination thereof. In some embodiments, a cancer therapy can comprise a CAR-T cell therapy, and the CAR-T therapy can be configured to target a biomarker of a cancer cell. In some embodiments, a biomarker can comprise CD30, CD20 CD19, CD22, CD138, CD44v6, CAIX, CEA, CD133, c-Met, EGFR, EGFRvIII, Epcam, EphA2, fetal acetylcholine receptor, FR alpha, GD2, GPC3, GUCY2C, HER1, HER2, ICAM-1, IL13R alpha 2, IL11R alpha, Kras, Kras G12D, L1CAM, MAGE, MET, mesothelin, MUC1, MUC16, NKG2D, NY-ESO-1, PSCA, WT-1, or any combination thereof. In some embodiments, a cancer therapy can comprise an immunotherapy. In some embodiments, an immunotherapy can comprise a checkpoint inhibitor. In some embodiments, a checkpoint inhibitor can comprise a CTLA-4 inhibitor, a PD-1 inhibitor, a PD-L1 inhibitor, a TIGIT inhibitor, a ATAR inhibitor, a B7-H3 inhibitor, a B7-H4 inhibitor, a BTLA inhibitor, an IDO inhibitor, a KIR inhibitor, a LAG inhibitor, a NOX2 inhibitor, a SIGLEC7 inhibitor, a SIGLEC9 inhibitor, a TIM3 inhibitor, a VISTA inhibitor, a biosimilar of any of these, or any combination thereof. In some embodiments, a checkpoint inhibitor can comprise pembrolizumab, nivolumab, ipilimumab, atezolizumab, avelumab, durvalumab, a salt of any of these, a biosimilar of any of these, or any combination thereof. In some embodiments, a cancer therapy can comprise a drug that can be licensed and approved for treatment of the cancer by a regulatory agency. In some embodiments, a cancer can comprise a glioblastoma, a pancreatic cancer, a breast cancer, a prostate cancer, an ovarian cancer, or any combination thereof. In some embodiments, a cancer can comprise a melanoma, a bladder cancer, a head or neck cancer, a kidney cancer, a liver cancer, a non-small cell lung cancer, or any combination thereof. In some embodiments, a malignant tumor can comprise a pancreatic tumor, a breast tumor, an ovarian tumor, a prostate tumor, a glioblastoma, or any combination thereof. In some embodiments, a malignant tumor can comprise a melanoma, a bladder tumor, a head tumor, a neck tumor, a kidney tumor, a liver tumor, a non-small cell lung tumor, or any combination thereof. In some embodiments, an allogeneic cell or a xenogeneic cell can be administered to or proximal to the malignant tumor. In some embodiments, administering of an allogeneic cell or a xenogeneic cell can be by parenchymal injection, intra-thecal injection, intra-ventricular injection, intra-cisternal injection, intratumoral injection, subcutaneous injection, intraperitoneal injection, a surgical route, or any combination thereof. In some embodiments, administering of an allogeneic cell or a xenogeneic cell can occur from about once a day to about once a year. In some embodiments, administering of an allogeneic cell or a xenogeneic cell can reduce a size of a tumor in a subject. In some embodiments, administering of the allogeneic cell or the xenogeneic cell can inhibit growth of a tumor in the subject. In some embodiments, an allogeneic cell or a xenogeneic cell can be an isolated and purified allogeneic cell or a xenogeneic cell. In some embodiments, an allogeneic cell or a xenogeneic cell may not comprise a hematopoietic cell. In some embodiments, an allogeneic cell or a xenogeneic cell can comprise a CAR T cell. In some embodiments, an allogeneic cell or a xenogeneic cell can comprise a B cell, a T cell, an NK cell, a macrophage, a dendritic cell, an endothelial cell, a stem cell, or any combination thereof. In some embodiments, an allogeneic cell or a xenogeneic cell can comprise an immune cell, a bone cell, a fat cell, a nerve cell, a skin cell, a pancreatic cell, a sex cell, a blood cell, a muscle cell, a stem cell or any combination thereof. In some embodiments, a tissue that comprises an allogeneic cell or a xenogeneic cell can be administered to the subject. In some embodiments, the method can further comprises administering an antibody, a peptide, an antibody drug conjugate, or any combination thereof. In some embodiments, the peptide can comprise an HLA or a portion thereof. In some embodiments, a method can further comprise performing an analysis on a sample comprising the allogeneic cell or the xenogeneic cell. In some embodiments, an analysis can comprise: performing a genetic assay on at least a portion of the sample; performing an epigenetic assay on at least a portion of the sample; obtaining at least a portion of a medical history of a donor of the sample; identifying a presence or absence of one or more cancer mutations in genetic material obtained from at least a portion of the sample, or any combination thereof. In some embodiments, wherein upon the administering the therapeutically effective amount of the allogeneic cell or the xenogeneic cell to the subject, there can be an increase in the number of immune cells to the site of the administering as measured by immunohistochemistry (IHC). In some embodiments, a method can further comprise selecting an allogeneic cell or a xenogeneic cell from a plurality of allogeneic cells or xenogeneic cells. In some embodiments, a subject can be a human subject. In some embodiments, a sample obtained from a subject may have been evaluated by a diagnostic test. In some embodiments, a diagnostic test can be an FDA approved or FDA cleared diagnostic test. In some embodiments, a subject can be a pediatric subject.

Also disclosed herein are cell banks comprising an allogeneic cell or a xenogeneic cell.

Also disclosed herein are kits comprising an allogeneic cell or a xenogeneic cell, a container, and instructions for use in administering the allogeneic cell or the xenogeneic cell to the subject to treat the cancer.

Also disclosed herein are pharmaceutical compositions comprising an allogeneic cell, a xenogeneic cell, or both in unit dose form and a pharmaceutically acceptable excipient, carrier, or diluent. In some embodiments, a pharmaceutical composition can be encapsulated. In some embodiments, a pharmaceutical composition can be in the form of an injectable liquid.

Also disclosed herein are methods of treating a subject having a cancer. In some embodiments, a method can comprise: administering a therapeutically effective amount of an isolated and purified HLA or a portion thereof to the subject, wherein the isolated and purified HLA or the portion thereof can be mismatched to an antigen marker profile of a cell of the subject. In some embodiments, the HLA or portion thereof can be a synthetic HLA or synthetic portion thereof. In some embodiments, the HLA or portion thereof can be a recombinant HLA or recombinant portion thereof. In some embodiments, more than one isolated and purified HLA or portions thereof can be administered to the subject. In some embodiments, a method can further comprise administering an allogeneic cell or a xenogeneic cell to a subject, wherein an antigen marker profile of a cell of the subject comprises at least one mismatched antigen marker as compared to an antigen marker profile of the allogeneic cell or a xenogeneic cell administered to the subject. In some embodiments, a cancer therapy can comprise: abemaciclib, abemaciclib, abiraterone acetate, abiraterone acetate, acalabrutinib, ado-trastuzumab emtansine, afatinib dimaleate, aldesleukin, alectinib, alemtuzumab, alpelisib, amifostine, aminolevulinic acid hydrochloride, anastrozole, apalutamide, aprepitant, arsenic trioxide, atezolizumab, avelumab, axicabtagene ciloleucel, axitinib, azacytidine, belinostat, bendamustine hydrochloride, bevacizumab, bexarotene, bicalutamide, binimetinib, bleomycin sulfate, blinatumomab, bortezomib, bosutinib, brentuximab vedotin, brigatinib, busulfan, cabazitaxel, cabozantinib-s-malate, calaspargase pegol-mknl, capecitabine, caplacizumab-yhdp, carboplatin, carfilzomib, carmustine, cemiplimab-rwlc, ceritinib, cetuximab, chlorambucil, cisplatin, cladribine, clofarabine, cobimetinib, copanlisib hydrochloride, crizotinib, cyclophosphamide, cytarabine, dabrafenib mesylate, dacarbazine, dacomitinib, dactinomycin, daratumumab, darbepoetin alfa, darolutamide, dasatinib, daunorubicin hydrochloride, decitabine, defibrotide sodium, degarelix, denileukin diftitox, denosumab, dexamethasone, dexrazoxane hydrochloride, dinutuximab, docetaxel, doxorubicin hydrochloride, durvalumab, duvelisib, elotuzumab, eltrombopag olamine, emapalumab-lzsg, enasidenib mesylate, encorafenib, entrectinib, enzalutamide, epirubicin hydrochloride, epoetin alfa, erdafitinib, eribulin mesylate, erlotinib hydrochloride, etoposide, everolimus, exemestane, fam-trastuzumab deruxtecan-nxki, fedratinib hydrochloride, filgrastim, fludarabine phosphate, fluorouracil, flutamide, fostamatinib disodium, fulvestrant, gefitinib, gemcitabine hydrochloride, gemcitabine-cisplatin, gemcitabine-oxaliplatin, gemtuzumab ozogamicin, gilteritinib fumarate, glasdegib maleate, glucarpidase, goserelin acetate, granisetron, granisetron hydrochloride, hydroxyurea, ibritumomab tiuxetan, ibrutinib, idarubicin hydrochloride, idelalisib, ifosfamide, imatinib mesylate, imiquimod, inotuzumab ozogamicin, iobenguane i131, ipilimumab, irinotecan hydrochloride, ivosidenib, ixabepilone, ixazomib citrate, lanreotide acetate, lapatinib ditosylate, larotrectinib sulfate, lenalidomide, lenvatinib mesylate, letrozole, leucovorin calcium, leuprolide acetate, lomustine, lorlatinib, lu 177-dotatate, mechlorethamine hydrochloride, megestrol acetate, melphalan, melphalan hydrochloride, mercaptopurine, methotrexate, methylnaltrexone bromide, midostaurin, mitomycin c, mitoxantrone hydrochloride, mogamulizumab-kpkc, moxetumomab pasudotox-tdfk, necitumumab, nelarabine, neratinib maleate, nilotinib, nilutamide, niraparib tosylate monohydrate, nivolumab, obinutuzumab, ofatumumab, olaparib, omacetaxine mepesuccinate, ondansetron hydrochloride, osimertinib mesylate, oxaliplatin, paclitaxel, palbociclib, palifermin, palonosetron hydrochloride, pamidronate disodium, panitumumab, panobinostat, pazopanib hydrochloride, pegaspargase, pegfilgrastim, peginterferon alfa-2b, pembrolizumab, pemetrexed disodium, pertuzumab, plerixafor, polatuzumab vedotin-piiq, pomalidomide, ponatinib hydrochloride, pralatrexate, prednisone, procarbazine hydrochloride, propranolol hydrochloride, radium 223 dichloride, raloxifene hydrochloride, ramucirumab, rasburicase, ravulizumab-cwvz, recombinant interferon alfa-2b, regorafenib, ribociclib, rituximab, rolapitant hydrochloride, romidepsin, romiplostim, rucaparib camsylate, ruxolitinib phosphate, selinexor, siltuximab, sipuleucel-t, sonidegib, sorafenib tosylate, sunitinib malate, tagraxofusp-erzs, talazoparib tosylate, talc, talimogene laherparepvec, tamoxifen citrate, temozolomide, temsirolimus, thalidomide, thioguanine, thiotepa, tisagenlecleucel, tocilizumab, topotecan hydrochloride, toremifene, trabectedin, trametinib, trastuzumab uridine, triacetate, valrubicin, vandetanib, vemurafenib, venetoclax, vincristine sulfate, vinorelbine tartrate, vismodegib, vorinostat, zanubrutinib, ziv-aflibercept, zoledronic acid, a pharmaceutically acceptable salt of any of these, or any combination thereof.

Also disclosed herein are methods of treating a subject having a cancer. In some embodiments, the subject is a subject in need thereof. In some embodiments, the method can comprise administering to the subject a therapeutically effective amount of: i) an allogeneic cell or a xenogeneic cell, ii) an isolated and purified HLA or a portion thereof, or iii) a combination of (i) and (ii) to the subject in need thereof, thereby treating the subject having the cancer. In some embodiments, an antigen marker profile of a cell of the subject can comprise at least one mismatched antigen marker as compared to an antigen marker profile of the allogeneic cell, a xenogeneic cell, or the isolated and purified HLA or the portion thereof, or both, administered to the subject. In some embodiments, the method can further comprise administering an immunotherapy, the immunotherapy can comprise a checkpoint inhibitor. In some embodiments, the allogeneic cell or the xenogeneic cell may not be genetically modified. In some embodiments, the allogeneic cell or the xenogeneic cell can comprise an adipose cell. In some embodiments, the subject can be a human and the allogeneic cell or the xenogeneic cell can be a human cell, a murine cell, a bovine cell, or a non-human primate cell or any combination thereof. In some embodiments, the cancer prior to the treating may not be responsive to administration of a monotherapy checkpoint inhibitor. In some embodiments, the checkpoint inhibitor can comprise a CTLA-4 inhibitor, a PD-1 inhibitor, a PD-L1 inhibitor, a TIGIT inhibitor, a ATAR inhibitor, a B7-H3 inhibitor, a B7-H4 inhibitor, a BTLA inhibitor, an IDO inhibitor, a KIR inhibitor, a LAG inhibitor, a NOX2 inhibitor, a SIGLEC7 inhibitor, a SIGLEC9 inhibitor, a TIM3 inhibitor, a VISTA inhibitor, a biosimilar of any of these, or any combination thereof. In some embodiments, the checkpoint inhibitor can comprise nivolumab, pembrolizumab, ipilimumab, atezolizumab, avelumab, cemiplimab, durvalumab, dostarlimab, a biosimilar of any of these, or any combination thereof. In some embodiments, the method can further comprise administering concurrently or consecutively an additional therapy. In some embodiments, the additional therapy can be a CAR-T therapy, a cancer vaccine therapy, or both. In some embodiments, the allogeneic, the xenogeneic cell, the isolated and purified HLA or a portion thereof, or any combination thereof, can be injected or delivered into the subject at a location proximal to the cancer. In some embodiments, the allogeneic cell, the xenogeneic cell, the isolated and purified HLA or a portion thereof, or any combination thereof, can be injected intratumorally into the subject. In some embodiments, the checkpoint inhibitor can be administered intravenously. In some embodiments, the allogeneic cell, the xenogeneic cell, the isolated and purified HLA or a portion thereof, or any combination thereof, can be administered concurrently or consecutively with the checkpoint inhibitor.

Also disclosed herein, are methods of treating a subject having a tumor. In some embodiments, a method can comprise: administering proximally, directly, or proximally and directly to the tumor of the subject a therapeutically effective amount of: i) an allogeneic cell, a xenogeneic cell, or both, ii) an isolated and purified HLA or a portion thereof, or iii) a combination of (i) and (ii). In some embodiments, a cell of the subject can comprise at least one mismatched HLA as compared to an HLA profile of the allogeneic cell, the xenogeneic cell, or the isolated and purified HLA or the portion thereof administered to the subject.

Also disclosed herein are methods of treating a PD-1 inhibitor or a PD-L1 inhibitor resistant tumor. In some embodiments, a method can comprise: administering proximally, directly, or proximally and directly to the tumor of the subject a therapeutically effective amount of: i) an allogeneic cell, a xenogeneic cell, or both, comprising at least one HLA mismatch as compared to an HLA profile of the subject, ii) an isolated and purified HLA or a portion thereof, wherein the isolated and purified HLA or a portion thereof is different than the HLA profile of the subject, or iii) a combination of (i) and (ii). In some embodiments, after the administering, a PD-1 inhibitor or a PD-L1 inhibitor resistant tumor can show at least in part an increased response to a PD-1 inhibitor or a PD-L1 inhibitor treatment relative to treatment with a PD-1 inhibitor or a PD-L1 inhibitor alone.

Also disclosed herein are methods of treating a subject having a cancer. In some embodiments, a method can comprise: administering a therapeutically effective amount of an xenogeneic cell to a subject. In some embodiments, an antigen marker profile of a cell of the subject can comprise at least one mismatched antigen marker as compared to an antigen marker profile of a xenogeneic cell administered to the subject. In some embodiments, the cancer can comprise a malignant tumor. In some embodiments, the antigen marker profile can comprise a human leukocyte antigen (HLA) or a portion thereof. In some embodiments, the at least one mismatched antigen marker can be 2 mismatched antigen markers. In some embodiments, the at least one mismatched antigen marker can be 3 mismatched antigen markers. In some embodiments, a cell transplant or tissue transplant comprising the xenogeneic cell can be administered to the subject. In some embodiments, a method can further comprise: administering an isolated and purified HLA or a portion thereof to the subject. In some embodiments, the isolated and purified HLA or the portion thereof can be mismatched to an antigen marker profile of a cell of the subject. In some embodiments, a method can further comprise, administering a cancer therapy to the subject. In some embodiments, the xenogeneic cell can be administered to or proximal to the malignant tumor. In some embodiments, the administering of the xenogeneic cell can be by parenchymal injection, intra-thecal injection, intra-ventricular injection, intra-cisternal injection, intratumoral injection, subcutaneous injection, intraperitoneal injection, a surgical route, or any combination thereof. In some embodiments, the xenogeneic cell can comprise an adipose cell.

Also disclosed herein are kits comprising a xenogeneic cell, a container, and instructions for use in administering the xenogeneic cell to the subject to treat the cancer.

Also disclosed herein are pharmaceutical compositions comprising a xenogeneic cell in unit dose form and a pharmaceutically acceptable excipient, carrier, or diluent.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows the mean tumor volume of a murine RM-1 prostate cancer model after treatment with: AIM (allo-immunotherapy), Anti-PD-1, AIM (allo-immunotherapy) and Anti-PD-1, and untreated vehicle (PBS control). The tumor volume was about 2100 mm³ in the dual AIM and Anti-PD-1 treatment group as compared to about 3500 mm³-3890 mm³ for the other three treatment groups.

FIG. 2 shows images of immune cell infiltrates into the AIM transplantation site at 3 days and 8 days post-implantation. The images are of 20× and 40× magnification. There was an increased immune influx from day 3 to day 8 post-implantation. Representative macrophages and granulocytes are indicated by the arrows.

### DETAILED DESCRIPTION

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. The description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The singular forms “a”, “an”, and “the” are used herein to include plural references unless the context clearly dictates otherwise. Accordingly, unless the contrary is indicated, the numerical parameters set forth in this application are approximations that can vary depending upon the desired properties sought to be obtained.

The terms “determining”, “measuring”, “evaluating”, “assessing,” “assaying,” and “analyzing” are often used interchangeably herein to refer to forms of measurement and include determining if an element may be present or not (for example, detection), or the amount of an element. These terms can include quantitative and qualitative determinations. Assessing can be alternatively relative or absolute. “Detecting the presence of” includes determining the amount of something present, as well as determining whether it may be present or absent.

The term “substantially” or “essentially” refers to a qualitative condition that exhibits an entire or nearly total range or degree of a feature or characteristic of interest. In some cases, substantially refers to at least about: 70%, 75%, 80%, 85%, 90%, 95%, 99%, 99.9% or 99.99% of the total range or degree of a feature or characteristic of interest. In some cases, the substantially or essentially refers to an amount that can be about 100% of a total amount.

The term “at least partially” refers to a qualitative condition that exhibits a partial range or degree of a feature or characteristic of interest. In some cases, at least partially refers to at least about: 5%, 10%, 20%, 30%, 40%, 50%, 60%, 70%, 75%, 80%, 85%, 90%, 95%, 99%, or 100% of the total range or degree of a feature or characteristic of interest.

Unless otherwise indicated, open terms for example “contain,” “containing,” “include,” “including,” and the like mean comprising.

As used herein, the term “about” or “approximately” means within an acceptable error range for the particular value as determined by one of ordinary skill in the art, which will depend in part on how the value is measured or determined, e.g., the limitations of the measurement system. For example, “about” means plus or minus 10%, per the practice in the art. Alternatively, “about” means a range of plus or minus 20%, plus or minus 10%, plus or minus 5%, or plus or minus 1% of a given value. Alternatively, particularly with respect to biological systems or processes, the term means within an order of magnitude, within 5-fold, or within 2-fold, of a value. Where particular values are described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges.

When used herein, a percentage of a material (e.g., a biological material, an excipient, a compound) of a composition is with respect to a total weight of a composition. In some cases, a percentage of a material of a composition is with respect to a total volume of a composition. In some cases, “Percentage by weight” or “w/w” means ratio of the mass of the specified ingredient verses the mass of the entire composition (e.g., dosage unit).

The term “subject,” “host,” “individual,” and “patient” are as used interchangeably herein to refer to an animal, typically mammalian animals. Any suitable mammal can be administered a composition as described herein or be treated by a method as described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). Mammals can be any age or at any stage of development, for example a mammal can be neonatal, infant, adolescent, adult or in utero. In some embodiments, a subject is a human. Humans can be more than about: 1, 2, 5, 10, 20, 30, 40, 50, 60, 65, 70, 75, 80, 85, 90, 95, 100, 105, 110, 115 or about 120 years of age. Humans can be less than about: 1, 2, 5, 10, 20, 30, 40, 50, 60, 65, 70, 75, 80, 85, 90, 95, 100, 105, 110, 115 or about 120 years of age. In some cases, a human can be less than about 18 years of age. In some cases, human can be from about 1 week to about 5 weeks old, 1 month to about 12 months old, from about 1 year to about 20 years, from about 15 years to about 50 years, from about 40 years to about 80 years, or from about 60 years to about 110 years. In some cases, a human can be more than about 18 years of age. A human may be a pediatric subject. A human may be an adult subject. A human can be a child subject. A mammal such as a human can be born a male or a female. In some embodiments, a subject can have or can be suspected of having a disease or condition, such as a cancer. The subject can be a patient, such as a patient being treated for a condition or a disease, such as a cancer. In some cases, a subject can be a responder to cancer therapy. In some cases, a subject can be a non-responder to a cancer therapy. A subject can be predisposed to a risk of developing a condition or a disease. A subject can be in remission from a condition or a disease. In some instances, a subject can be healthy. A subject may be a subject in need thereof. A subject may have received a positive diagnosis of the cancer. A subject may have received a cancer therapy that failed to treat a cancer.

A “therapeutically effective amount” refers to an amount of a composition as disclosed herein with or without additional agents that is effective to achieve its intended purpose, for example to treat a disease. Individual patient needs may vary. Generally, the dosage required to provide an effective amount of the composition will vary, depending on the age, health, physical condition, sex, weight, extent of the disease of the recipient, frequency of treatment and the nature and scope of the disease or condition.

As used herein, the terms “treatment” or “treating” refers to a pharmaceutical or other intervention regimen for obtaining beneficial or desired results in the recipient. Beneficial or desired results include but are not limited to a therapeutic benefit and/or a prophylactic benefit. A therapeutic benefit refers to eradication or amelioration of one or more symptoms of an underlying disorder being treated. For example, a therapeutic benefit can comprise shrinking a tumor, at least partially inhibiting a tumor to spread, reducing the size of a tumor, inhibiting a tumor to grow, slowing tumor growth, or any combination thereof. Also, a therapeutic benefit can be achieved with the eradication or amelioration of one or more of the physiological symptoms associated with the underlying disorder such that an improvement may be observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For a prophylactic benefit, a subject at risk of developing a particular disease, or a subject reporting one or more of the physiological symptoms of a disease may undergo a treatment disclosed herein, even though a diagnosis of this disease may not have been made.

The section headings used herein are for organizational purposes only and are not to be construed as limiting the subject matter described

### Overview

Disclosed herein are compositions and methods for treating cancer. Allo-immunotherapy (AIM) as described herein can harness the transplant rejection response to prime the immune system to target a cancer. For example, an allo-immunotherapy can be used to target an immune response to a tumor microenvironment. In some cases, AIM therapy can comprise an allogeneic cell, a xenogeneic cell, or a combination of both. For example, an AIM can increase the amount of immune cells by at least about: 2 fold to about 100 fold, 10 fold to about 1000 fold, or 100 fold to about 1000 fold, at the site of a tumor as compared to the tumor prior to AIM. In another example, an AIM can induce a 100-1000 fold stronger immune response than a nominal antigen such as a virus. An AIM can active the immune system with an alloantigen or xenoantigen. In some instances, AIM can localize, and reprogram immune cells at a tumor site. The compositions disclosed herein can be used individually or in combination with other cancer therapeutics to treat cancers. Allo-immunotherapy compositions can comprise a cell, a tissue, a biological material such as a protein, or a combination thereof. Allogeneic transplantation comprises transplantation of a cell, a tissue, or a biological material with a mismatched HLA antigen. For example, allogeneic transplantation can comprise allogeneic transplantation of a cell or a tissue with one or more mismatched Human Leukocyte Antigens (HLA). Allo-immunotherapy can increase an immune response to tumor environment and thus stimulate an increased anti-cancer response. In some instances, AIM can be used with other cancer therapies, such as a checkpoint inhibitor, to increase the effectiveness of the checkpoint inhibitor and/or AIM therapy.

The HLA molecule or allogeneic cell may comprise an HLA-mismatch as compared to the subject receiving the composition. The composition may recruit endogenous or transplanted immune cells to the tumor site and may activate the recipient's immune system. In some cases, recruitment of immune cells to a tumor site may activate the tumor microenvironment, likened to a “hot” tumor microenvironment. In some cases, an activated tumor microenvironment may cause a tumor to shrink or may eliminate a tumor. In other instances, an activated tumor microenvironment may cause a tumor to grow more slowly than a tumor would grow in the absence of an activated tumor microenvironment. The recipient may be treated with a checkpoint inhibitor, or another immune activating immunotherapy, to substantially eliminate the tumor. For example, after a patient has been treated with allo-immunotherapy such as an allogeneic cell or an HLA molecule the patient can be treated with a checkpoint inhibitor. In some cases, a targeted immune response to alloantigen can improve an immune system response by activating one or more immune cells in the tumor microenvironment, shifting a cold, immune-deficient tumor microenvironment to a hot, immune-rich environment. Subjects having a hot tumor may achieve successful outcomes when treated with a checkpoint inhibitor immunotherapy or a checkpoint inhibitor immunotherapy with a AIM.

Allogeneic and/or Xenogeneic Cell and Tissue Treatment

Compositions disclosed herein can comprise an allogeneic cell or an allogeneic tissue. In some cases, composition disclosed herein can comprise a xenogeneic cell or a xenogeneic tissue. In some cases, composition disclosed herein can comprise a xenogeneic cell, a xenogeneic tissue, an allogeneic cell, a allogeneic tissue, or any combination thereof. In some embodiments, an allogeneic tissue can comprise one or more types of allogeneic cells. In some embodiments, an xenogeneic tissue can comprise one or more types of xenogeneic cells. For example, an allogeneic tissue can comprise 1, 2, 3, 4, 5, or more different types of allogeneic cells. An allogeneic cell or tissue can be obtained by a donor. In some cases, a donor can be related or may not be related to a subject. In some instances, a composition can comprise more than one allogeneic cell type. For example, a composition disclosed herein can comprise 1, 2, 3, 4, 5, or more different types of allogeneic cells. An allogeneic cell may be obtained from any cell source, tissue source, or organ source. In some cases, an allogeneic cell or tissue can comprise one or more mismatched antigen markers. For example, an allogeneic cell can comprise 1, 2, 3, 4, 5, 6, or more mismatched antigen markers. In some cases, an antigen marker can comprise an HLA. In some embodiments, a cell bank or another medical facility can comprise a composition herein, an allogeneic cell disclosed herein, an allogeneic tissue disclosed herein, an HLA molecule or a portion thereof disclosed herein, or any combination thereof. In some embodiments, an allogeneic cell can comprise a xenogeneic cell. For example, an allogeneic cell herein can comprise a pig cell, a primate cell, a mouse cell, or any animal cell (e.g., a mammalian cell). In some embodiments, an allogeneic cell can comprise necrotic cell.

In some embodiments, an allogeneic cell or a xenogeneic cell can comprise an immune cell, a bone cell, a fat cell, a nerve cell, a skin cell, a pancreatic cell, a sex cell, a blood cell, a muscle cell, a stem cell, or any combination thereof. In some cases, an allogeneic cell or a xenogeneic cell may be an immune cell. In some cases, an allogeneic cell or a xenogeneic cell can be a B-cell, a T-cell, an NK cell, a macrophage, a dendritic cell, a monocyte, a granulocyte, an eosinophil, a neutrophil, a basophil, a lymphocyte, a mast cell, or any combination thereof. In some cases, an allogeneic cell can comprise a CD4+ T cell, a CD8+ T cell, a T_{fh} cell, a Th22 cell, a Th17 cell, a Th1 cell, a Th2 cell, a T_{reg} cell, a Th9 cell, or a combination thereof. In some cases, an allogeneic cell or a xenogeneic cell can comprise a memory cell, such as a memory T-cell or a memory B-cell. In some cases, an allogeneic cell or a xenogeneic cell can comprise a stem cell, such as an embryonic stem cell, a tissue-specific stem cell, a mesenchymal stem cell, an induced pluripotent stem cell (IPSC), or a combination thereof. In some cases, an allogeneic cell or a xenogeneic cell can comprise a bone cell, such as an osteogenic cell, an osteoblast, an osteoclast, an osteocyte, or a combination thereof. In some cases, an allogeneic cell or a xenogeneic cell can comprise a blood cell such as a red blood cell, a platelet, or a combination thereof. In some cases, an allogeneic cell or a xenogeneic cell can comprise a muscle cell, such as a skeletal muscle cell, a cardiac muscle cell, a smooth muscle cell, or a combination thereof. In some cases, an allogeneic cell or a xenogeneic cell can comprise a skin cell, such as a keratinocyte, a melanocyte, a Langerhans cell, a Merkel cell, a fibroblast, a squamous cell, a basal cell, or a combination thereof. In some cases, an allogeneic cell or a xenogeneic cell can comprise a fat cell, such as an adipocyte. In some cases, a fat cell can comprise a white fat cell, a brown fat cell, a marrow fat cell, or a combination thereof. In some cases, a fat cell can comprise a subcutaneous fat cell. In some cases, an allogeneic cell or a xenogeneic cell can comprise a nerve cell, such as a neuron, a glial cell, or a combination thereof. In some cases, a neuron can comprise, a sensory neuron, a motor neuron, a pyramidal neuron, an astrocyte, a Betz cell, a microglia, or any combination thereof. In some cases, an allogeneic cell or a xenogeneic cell can comprise an endothelial cell. In some cases, an allogeneic cell or a xenogeneic cell can be an epithelial cell. In some cases, an allogeneic cell or a xenogeneic cell can be a cell of an organ such as a digestive organ (e.g., a stomach cell, an intestinal cell). In some cases, an allogeneic cell or a xenogeneic cell can comprise a pancreatic cell, such as an alpha cell, a beta cell, a delta cell, an F cell, or a combination thereof. In some cases, an allogeneic cell can comprise a sex cell, such as a sperm, an egg, or any combination thereof. In some cases, an allogeneic cell may be a genetically altered immune cell, such as a chimeric antigen receptor (CAR) cell (e.g., CAR T-Cell). In some cases, an allogeneic cell or a xenogeneic cell may be a bone marrow cell, such as a bone marrow progenitor cell, a hematopoietic cell, a marrow adipose cell, a stromal cell, or a combination thereof. In some instances, an allogeneic cell or a xenogeneic cell may not comprise a hematopoietic cell.

In some embodiments, a method herein can comprise selecting one or more allogeneic cells from a plurality of allogeneic cells. In some cases, a method herein can select 1, 2, 3, 4, 5, 6, or more allogeneic cell types from a plurality of cell types. In some embodiments, a method herein can comprise selecting one or more xenogeneic cells from a plurality of xenogeneic cells. In some cases, a method herein can select 1, 2, 3, 4, 5, 6, or more xenogeneic cell types from a plurality of cell types. In some cases, the selecting can comprise a cell sorting method (e.g., flow cytometry), a serological assay, a microscopy based method, or any combination thereof.

In some embodiments, a composition herein can comprise an allogeneic tissue or a xenogeneic tissue. In some cases, an allogeneic tissue or a xenogeneic tissue can comprise an allogeneic cell or a xenogeneic cell described herein. In some cases, an allogeneic tissue or a xenogeneic tissue can comprise a portion of a tissue. In some instances, an allogeneic tissue or a xenogeneic tissue can comprise an epithelial tissue, a connective tissue, a muscle tissue, a nervous tissue, or a mixture thereof. In some cases, a tissue can comprise a tissue of an organ. In some cases, a tissue of an organ can comprise a portion of an organ. For example, a tissue can comprise an integumentary tissue, a skeletal tissue, a muscular tissue, a circulatory tissue, a respiratory tissue, a digestive tissue, a urinary tissue, an immune tissue, a nervous tissue, an endocrine tissue, a reproductive tissue, or a combination thereof. In some embodiments, an allogeneic tissue can comprise a xenogeneic tissue. In some embodiments, an allogeneic tissue can comprise necrotic tissue.

In some instances, the allogeneic cell or a xenogeneic cell can comprise one or more mismatched antigen markers. In some cases, a donor can be related or may not be related to a subject. In some instances, a composition can comprise more than one allogeneic cell type or xenogeneic cell type. For example, a composition disclosed herein can comprise 1, 2, 3, 4, 5, or more different types of allogeneic cells. An allogeneic cell or a xenogeneic cell may be obtained from any cell source, tissue source, or organ source

In some embodiments, an allogeneic cell or a xenogeneic cell obtained from a donor may comprise a partially or fully HLA-mismatch as compared to the subject receiving the treatment. Human Leukocyte Antigens (HLA) are proteins that can be located on the surface of the different cell types. In some instances, there can be three groups of HLA, which are HLA-A, HLA-B and HLA-DR. A subject's HLA type is inherited as a set of the three HLA groups-A, B, and DR. Within the three groups there are many different specific HLA proteins, for example, there are over 50 different HLA-A proteins, 115 different HLA-B proteins and 120 different HLA-DR proteins. In some cases, each of these HLA has a different numerical designation, for example, a donor allogeneic cell may have HLA-A3, while a subject to receive a therapy can have HLA-A2. An allogeneic cell described herein can comprise at least one mismatch within the three HLA groups, for example an allogeneic cell can comprise a mismatch at HLA-A, HLA-B or HLA-DR to a subject's HLA type. In some cases, an allogeneic cell can comprise a mismatch at one HLA group, at two HLA groups, or at all three HLA groups. In some cases, a genetic test, such as sequencing can be used to determine a subject or donors HLA type. In some cases, a serological assay can be used to determine a subject or donor HLA type.

Disclosed herein are methods of performing an analysis on a sample containing an allogeneic cell, a tissue, an HLA molecule or a portion thereof, or any combination thereof described herein. In some cases, an analysis can comprise performing a genetic assay on at least a portion of the sample; performing an epigenetic assay on at least a portion of the sample; obtaining at least a portion of a medical history of a donor of the sample; identifying a presence or absence of one or more cancer mutations in genetic material obtained from at least a portion of the sample, or any combination thereof. In some cases, a diagnostic test can be performed on an sample from a subject. For example, a diagnostic test for a disease or condition can be performed. In some cases, a diagnostic test can comprise a polymerase chain reaction (PCR) test, a sequencing test (e.g., next generation sequencing assay), a serological assay, an enzyme-linked immunoassay (ELISA), or a combination thereof. In some cases, a diagnostic test can be an FDA approved or an FDA cleared diagnostic test.

In some embodiments, a treatment herein can comprise a prime and/or boosting method. In some cases, a prime/boosting method can comprise administering an allogeneic cell, a xenogeneic cell, a portion thereof, or any combination thereof to a subject prior to implanting an allogeneic cell, a xenogeneic cell, a portion thereof, or any combination thereof in a subject. In some cases, administration can comprise intravenous administration of the allogeneic cell, the xenogeneic cell, a portion thereof, or any combination thereof. In some cases, the administration can be at least 1 day, 2 days, 3 days, 4 days, 5 days, 6 days, 7 days, 8 days, 9 days, 10 days, 11 days, 12 days, 13 days, 14 days, 16 days, 18 days, 21 days, or more than 21 days prior the implanting of the allogeneic cell, the xenogeneic cell, a portion thereof, or any combination thereof.

### HLA Molecule Treatment and Other AIM Treatments

Compositions disclosed herein can comprise an HLA or a portion thereof. In some cases, an HLA or a portion thereof can be an isolated and purified HLA or a portion thereof. An isolated and purified HLA can be a recombinant HLA or a recombinant portion thereof. For example, an HLA can be expressed and purified from a cell. In some cases, an HLA molecule can be a synthetic molecule. In some cases, an isolated and purified HLA can be administered individually, with an allogeneic cell, with a cancer therapy, or any combination thereof.

Compositions disclosed herein can comprise an antibody that can induce an HLA-mismatch immune response. Compositions disclosed herein can comprise a peptide (e.g., a protein) that can induce an HLA-mismatch immune response. Compositions disclosed herein can comprise an antibody drug conjugate that can induce an HLA-mismatch immune response. Compositions disclosed herein can comprise a cell therapy that can induce an HLA-mismatch immune response. Compositions disclosed herein can comprise a molecule that can mimic an HLA-mismatch. For example, a liposome can be configured to express a mismatched HLA-protein.

### Pharmaceutical Compositions

In some embodiments, disclosed herein are compositions that can comprise an HLA or a portion thereof, an isolated and purified HLA, an allogeneic cell, an allogeneic tissue, a xenogeneic cell, a xenogeneic tissue, an antigenic marker or a portion thereof, a cancer therapy, or any combination thereof. In some embodiments, a composition herein can comprise a pharmaceutical composition. For example, a pharmaceutical composition herein can comprise an HLA or a portion thereof, an isolated and purified HLA, an allogeneic cell, an allogeneic tissue, an antigenic marker or a portion thereof, a cancer therapy, or any combination thereof. In some embodiments, compositions herein such as pharmaceutical compositions can comprise an excipient, a carrier, a diluent or any combination thereof. In some cases, an excipient, a carrier, or a diluent can be a pharmaceutically acceptable excipient, a pharmaceutically acceptable carrier or a pharmaceutically acceptable diluent.

As used herein, reference to a therapy, a compound, or a composition, includes reference to any salt, solvate, ester, or polymorph of the therapy, the compound, or the composition. A “salt” can include a pharmaceutically acceptable salt. Examples of pharmaceutically acceptable salts can include those salts prepared by reaction of a compound disclosed herein with a mineral, organic acid or inorganic base, such salts including, acetate, acrylate, adipate, alginate, aspartate, benzoate, benzenesulfonate, bisulfate, bisulfite, bitartrate, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate. metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undeconate, and xylenesulfonate. Further, a compound disclosed herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, Q-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. Other acids, such as oxalic, while not in themselves pharmaceutically acceptable, can be employed in the preparation of salts useful as intermediates in obtaining a compound and/or a pharmaceutically acceptable acid addition salt. In some embodiments, a compound disclosed herein which can comprise a free acid group reacts with a suitable base, such as the hydroxide, carbonate, bicarbonate, sulfate, of a pharmaceutically acceptable metal cation, with ammonia, or with a pharmaceutically acceptable organic primary, secondary or tertiary amine. Representative alkali or alkaline earth salts can include the lithium, sodium, potassium, calcium, magnesium, and aluminum salts and the like. Illustrative examples of bases can include sodium hydroxide, potassium hydroxide, choline hydroxide, sodium carbonate, N+(C1-4 alkyl)4, and the like. Representative organic amines useful for the formation of base addition salts can include ethylamine, diethylamine, ethylenediamine, ethanolamine, diethanolamine, piperazine and the like. It may be understood that a compound disclosed herein can also include the quaternization of any basic nitrogen-containing groups they contain. In some embodiments, water or oil-soluble or dispersible products can be obtained by such quaternization. A compound disclosed herein can be prepared as pharmaceutically acceptable salts formed when an acidic proton present in the parent compound either can be replaced by a metal ion, for example an alkali metal ion, an alkaline earth ion, or an aluminum ion; or coordinates with an organic base. In some embodiments, base addition salts can be also prepared by reacting the free acid form of a compound disclosed herein with a pharmaceutically acceptable inorganic or organic base, including, but not limited to organic bases such as ethanolamine, diethanolamine, triethanolamine, tromethamine, N-methylglucamine, and the like and inorganic bases such as aluminum hydroxide, calcium hydroxide, potassium hydroxide, sodium carbonate, sodium hydroxide, and the like. In addition, the salt forms of the disclosed compounds can be prepared using salts of the starting materials or intermediates.

In some embodiments, a composition can comprise an excipient. In some embodiments, a composition herein can comprise one or more of the following excipients: acacia, acesulfame potassium, acetic acid-glacial, acetone, acetyltributyl citrate, acetyltriethyl citrate, adipic acid, agar, albumin, alcohol, alginic acid, aliphatic polyesters, alitame, almond oil, alpha tocopherol, aluminum hydroxide adjuvant, aluminum monostearate, aluminum oxide, aluminum phosphate adjuvant, ammonia solution, ammonium alginate, ammonium chloride, ascorbic acid, ascorbyl palmitate, aspartame, attapulgite, bentonite, benzalkonium chloride, benzethonium chloride, benzoic acid, benzyl alcohol, benzyl benzoate, boric acid, bronopol, butylated hydroxyanisole, butylated hydroxytoluene, butylene glycol, butylparaben, calcium acetate, calcium alginate, calcium carbonate, calcium chloride, calcium hydroxide, calcium lactate, calcium phosphate-dibasic anhydrous, calcium phosphate-dibasic dihydrate, calcium phosphate-tribasic, calcium silicate, calcium stearate, calcium sulfate, canola oil, carbomer, carbon dioxide, carboxymethylcellulose calcium, carboxymethylcellulose sodium, carrageenan, castor oil, castor oil-hydrogenated, cellulose-microcrystalline, cellulose-microcrystalline and carboxymethylcellulose sodium, cellulose-powdered, cellulose-silicified microcrystalline, cellulose acetate, cellulose acetate phthalate, ceratonia, ceresin, cetostearyl alcohol, cetrimide, cetyl alcohol, cetylpyridinium chloride, chitosan, chlorhexidine, chlorobutanol, chlorocresol, chlorodifluoroethane (hcfc), chlorofluorocarbons (cfc), chloroxylenol, cholesterol, citric acid monohydrate, coconut oil, colloidal silicon dioxide, coloring agents, copovidone, corn oil, corn starch and pregelatinized starch, cottonseed oil, cresol, croscarmellose sodium, crospovidone, cyclodextrins, cyclomethicone, denatonium benzoate, dextrates, dextrin, dextrose, dibutyl phthalate, dibutyl sebacate, diethanolamine, diethyl phthalate, difluoroethane (hfc), dimethicone, dimethyl ether, dimethyl phthalate, dimethyl sulfoxide, dimethylacetamide, disodium edetate, docusate sodium, edetic acid, erythorbic acid, erythritol, ethyl acetate, ethyl lactate, ethyl maltol, ethyl oleate, ethyl vanillin, ethylcellulose, ethylene glycol stearates, ethylene vinyl acetate, ethylparaben, fructose, fumaric acid, gelatin, glucose-liquid, glycerin, glyceryl behenate, glyceryl monooleate, glyceryl monostearate, glyceryl palmitostearate, glycine, glycofurol, guar gum, hectorite, heptafluoropropane (hfc), hexetidine, hydrocarbons (hc), hydrochloric acid, hydrophobic colloidal silica, hydroxyethyl cellulose, hydroxyethylmethyl cellulose, hydroxypropyl betadex, hydroxypropyl cellulose, hydroxypropyl cellulose-low-substituted, hydroxypropyl starch, hypromellose, hypromellose acetate succinate, hypromellose phthalate, imidurea, inulin, iron oxides, isomalt, isopropyl alcohol, isopropyl myristate, isopropyl palmitate, kaolin, lactic acid, lactitol, lactose-anhydrous, lactose-inhalation, lactose-monohydrate, lactose-monohydrate and corn starch, lactose-monohydrate and microcrystalline cellulose, lactose-monohydrate and povidone, lactose-monohydrate and powdered cellulose, lactose-spray-dried, lanolin, lanolin-hydrous, lanolin alcohols, lauric acid, lecithin, leucine, linoleic acid, macrogol 15 hydroxystearate, magnesium aluminum silicate, magnesium carbonate, magnesium oxide, magnesium silicate, magnesium stearate, magnesium trisilicate, maleic acid, malic acid, maltitol, maltitol solution, maltodextrin, maltol, maltose, mannitol, medium-chain triglycerides, meglumine, menthol, methionine, methylcellulose, methylparaben, mineral oil, mineral oil-light, mineral oil and lanolin alcohols, monoethanolamine, monosodium glutamate, monothioglycerol, myristic acid, myristyl alcohol, neohesperidin dihydrochalcone, neotame, nitrogen, nitrous oxide, octyldodecanol, oleic acid, oleyl alcohol, olive oil, palmitic acid, paraffin, peanut oil, pectin, pentetic acid, petrolatum, petrolatum and lanolin alcohols, phenol, phenoxyethanol, phenylethyl alcohol, phenylmercuric acetate, phenylmercuric borate, phenylmercuric nitrate, phospholipids, phosphoric acid, polacrilin potassium, poloxamer, polycarbophil, polydextrose, poly (dl-lactic acid), polyethylene glycol, polyethylene oxide, polymethacrylates, poly(methyl vinylether/maleic anhydride), polyoxyethylene alkyl ethers, polyoxyethylene castor oil derivatives, polyoxyethylene sorbitan fatty acid esters, polyoxyethylene stearates, polyoxylglycerides, polyvinyl acetate phthalate, polyvinyl alcohol, potassium alginate, potassium alum, potassium benzoate, potassium bicarbonate, potassium chloride, potassium citrate, potassium hydroxide, potassium metabisulfite, potassium sorbate, povidone, propionic acid, propyl gallate, propylene carbonate, propylene glycol, propylene glycol alginate, propylparaben, propylparaben sodium, pyrrolidone, raffinose, saccharin, saccharin sodium, safflower oil, saponite, sesame oil, shellac, simethicone, sodium acetate, sodium alginate, sodium ascorbate, sodium benzoate, sodium bicarbonate, sodium borate, sodium carbonate, sodium chloride, sodium citrate dihydrate, sodium cyclamate, sodium formaldehyde sulfoxylate, sodium hyaluronate, sodium hydroxide, sodium lactate, sodium lauryl sulfate, sodium metabisulfite, sodium phosphate-dibasic, sodium phosphate-monobasic, sodium propionate, sodium starch glycolate, sodium stearyl fumarate, sodium sulfite, sodium thiosulfate, sorbic acid, sorbitan esters (sorbitan fatty acid esters), sorbitol, soybean oil, starch, starch-pregelatinized, starch-sterilizable maize, stearic acid, stearyl alcohol, sucralose, sucrose, sucrose octaacetate, sugar-compressible, sugar-confectioner's, sugar spheres, sulfobutylether b-cyclodextrin, sulfur dioxide, sulfuric acid, sunflower oil, suppository bases-hard fat, tagatose, talc, tartaric acid, tetrafluoroethane (hfc), thaumatin, thimerosal, thymol, titanium dioxide, tragacanth, trehalose, triacetin, tributyl citrate, tricaprylin, triethanolamine, triethyl citrate, triolein, vanillin, vegetable oil-hydrogenated, vitamin e polyethylene glycol succinate, water, wax-anionic emulsifying, wax-carnauba, wax-cetyl esters, wax-microcrystalline, wax-nonionic emulsifying, wax-white, wax-yellow, xanthan gum, xylitol, zein, zinc acetate, and/or zinc stearate.

In some cases, a composition can herein can comprise a detackifier, an anti-foaming agent, a buffering agent, a polymer, an antioxidant, a preservative, a chelating agent, a viscomodulator, a tonicifier, a flavorant, a colorant, an odorant, an opacifier, a suspending agent, a binder, a filler, a plasticizer, a lubricant, or any combination thereof.

In some cases, a composition such as a pharmaceutical composition can comprise a carrier or a diluent. In some instances, a carrier or diluent can comprise a water, an alcohol, a salt solution (e.g., saline), or a mixture thereof. In some instances, a carrier can comprise a carbohydrate, a buffer, a salt, a pH adjuster, or any combination thereof. In some cases, sodium phosphate, citric acid, acetic acid, tromethamine, histidine, gluconic, lactic acid, tartaric acid, aspartic acid, glutamic acid, a citric acid cycle intermediate, or any combination thereof can be a buffer. In some cases, a carrier can be a substrate used in the process of drug delivery. In some cases, a carrier can contribute a product's attributes such as stability, biopharmaceutical profile, appearance, and patient acceptability. In some cases, a carrier can be an organic excipient.

### Methods of Treatment

Disclosed herein are methods for treating cancer in a subject in need thereof. In some cases, methods may employ delivery of an HLA molecule (such as a synthetic or purified HLA molecule), a cell, a tissue, or a combination thereof to a subject with a cancer. In some cases, a method can comprise delivering an antibody, a peptide, an antibody drug conjugate, a protein, a cell therapy or a molecule that can mimic an HLA-mismatch. In some cases, a method of treating a cancer can comprise administering a xenogeneic cell, a xenogeneic tissue, an allogeneic cell, a allogeneic tissue, or any combination thereof. The HLA molecule or the allogeneic cell and/or tissue obtained from a donor may comprise a partially or fully HLA-mismatch as compared to the subject receiving the treatment. Methods may include delivering a composition (such as an HLA molecule or allogeneic cell) to a tumor site, proximal a tumor site, or both proximal and direct application to a tumor site. In some instances, the composition can be delivered proximal and directly to a tumor site. Methods may include delivering a composition (such as an HLA molecule or allogeneic cell) to the site of a cancer, proximal the site of a cancer, or both proximal and direct application to the site of a cancer. In some cases, administration of a composition herein can reduce the size of a tumor in a subject. An additional treatment, such as a cancer therapy may be administered. The cancer therapy may include an immunotherapy, such as a checkpoint immunotherapy. Also disclosed herein, are compositions for use in the treatment of a disease, for example for use in the treatment of a cancer. In some embodiments, disclosed herein is the use of a composition for the manufacture of a medicament for the treatment of cancer. An example composition can comprise an allogeneic cell or an isolated and purified HLA, and an excipient, a diluent, a carrier or any combination thereof.

In some embodiments, a method can comprise treating a subject having a cancer. In some embodiments, the method can comprise administering a therapeutically effective amount of an allogeneic cell and/or a xenogeneic cell to a subject. In some cases, an antigen marker profile of a cell of the subject can comprise at least one mismatched antigen marker as compared to an antigen marker profile of the allogeneic cell or the isolated and purified HLA or a portion thereof administered to the subject. In some cases, an antigen marker profile of a cell of the subject can comprise at least two or three mismatched antigen marker as compared to an antigen marker profile of the allogeneic cell administered to the subject. In some embodiments, the method can comprise administering a therapeutically effective amount of an isolated and purified HLA or a portion thereof to a subject. In some cases, an isolated and purified HLA or a portion thereof is mismatched to an antigen marker profile of a cell of the subject. In some instances, an HLA or portion thereof can be a synthetic HLA or synthetic portion thereof. In some instances, an HLA or portion thereof can be a recombinant HLA or recombinant portion thereof. In some cases, more than one isolated and purified HLA or portions thereof can be administered to a subject. For example, 2, 3, 4, 5, 6, or more purified HLAs or portions thereof can be administered to a subject.

Delivery of a cell, a tissue, an isolated and purified HLA, or a portion thereof can comprise an allo-immunotherapy (AIM). In some cases, delivery of a mismatched antigen marker, such as a synthetic HLA can comprise an AIM. Methods may include treating a cancer patient by delivering to a tumor site (i.e., intratumoral), proximal to a tumor site, or both: (i) a cell transplant (such as an allogeneic cell), (ii) an HLA molecule or portion thereof (such as a recombinant HLA), or (iii) a combination thereof. Methods may include recruitment of immune cells (endogenous or co-delivered) to the tumor thereby at least partially eliminating non-self/allogeneic antigen that may result in direct and indirect tumor immunity. In some cases, the immune response to alloantigen can be 100-1000× as strong as the response to a nominal antigen (e.g., an influenza virus). In some cases, the immune response in a tumor environment to an alloantigen treatment can be about 10 times to about 1000 times as strong as the response in a tumor environment without treatment of an alloantigen. In some cases, the immune response in a tumor environment to an alloantigen treatment can be about: 2 times to about 5 times, 3 time to about 8 times, 5 times to about 10 times, 8 times to about 15 times, 10 times to about 20 times, 15 times to about 30 times, 30 times to about 60 times, 50 times to about 100 times, 100 times to about 500 times, 250 times to about 750 times or about 500 times to about 1000 times as strong as the response in a tumor environment without treatment of an alloantigen. In some cases, a response can be measured by measuring the number of immune cells in a tumor environment. In some instances, measuring can be by counting via microscopy, by flow cytometry, immunohistochemistry (IHD) or a combination thereof. In some cases, a response can be measured by measuring the amount of cytokines in a tumor environment. In some cases, the amount of cytokines can be measured by an enzyme-linked immunosorbent assay (ELISA), or similar protein measuring assay.

In some embodiments, administering an effective amount of an allogeneic cell or a xenogeneic cell to a subject can induce an influx (e.g., an increase in a number) of immune cells to the site of the administering. In some cases, an influx of immune cells can be more than about, less than about, or equal to a: 10%, 20%, 30%, 40%, 50%, 60%, 70%, 80%, 90%, 100%, 110%, 120%, 130%, 140%, 150%, 160%, 170%, 180%, 190%, or 200% increase in the amount of the immune cells at the site of the administering as compared to the site prior to the administering. In some cases, an influx of immune cells can be an increase of about: 10% to about 200%, 10% to about 100%, 10% to about 50%, 10% to about 30%, 20% to about 40%, 40% to about 90%, 50% to about 120%, 70% to about 150%, 80% to about 100%, or 130% to about 200% in the amount of the immune cells at the site of the administering as compared to the site prior to the administering. In some cases, an influx of immune cells can be measured at about: 1 day, 2 days, 3 days, 4 days, 5 days, 6 days, 7 days, 8 days, 9 days, 10 days, 11 days, 12 days, 13 days, 14 days, or more than 14 days after the administration or after about: 1 day, 2 days, 3 days, 4 days, 5 days, 6 days, 7 days, 8 days, 9 days, 10 days, 11 days, 12 days, 13 days, 14 days, or more than 14 days post administration. In some cases, the influx of immune cells can be measured by flow cytometry, microscopy or any method for determining cell count.

In some embodiments, AIM can trigger a response to an alloantigen (e.g., similar to transplant rejection) and serve as bait for the immune system. For example AIM can attract allo-antigen-activated immune cells to the AIM transplant and, thus, the co-localized tumor. In some cases, an AIM can transform a tumor microenvironment to an immune-cell-rich, “hot” environment. In some embodiments, AIM can 1) bolster tumor penetration 2) sensitize the tumor to standard of care chemotherapy, and other systemic agents 3) act as an inflammatory target to increase the ability of host immune cells and CAR-T (B, NK, etc.) therapies to traffic to the tumor or 4) any combination of 1-3.

In some embodiments, AIM, which can be comprised of an MHC mismatched alloantigen, can lure the immune system to the site of a tumor to generate an inflammatory, immune-rich environment. In some embodiments, AIM can be combined with a standard of care therapy and other immune-activating therapies to extend overall survival for patients with cancer. An alloantigen can elicit a robust immune response compared with the response to a nominal antigen, such as the flu. For example, a T-cell precursor frequency for an alloantigen can be 5% of the T-cell population, which is 100-1000× higher compared to the T-cell precursor frequency for a nominal antigen (0.05-0.005%). An MHC-mismatched AIM therapy can drive acute rejection, and thus induce an influx of immune cells at the site of the tumor to shift from an immune-poor to an immune-rich tumor microenvironment. In some cases, an alloantigen can activates danger signals and/or Pathogen-associated molecular patterns (PAMPs) via the innate immune system to mature antigen-presenting cells (APCs) and provide a second inflammatory signal to activate the adaptive immune response and cytotoxic tumor-killing. The influx of immune cells to the tumor microenvironment can increase antigen presentation and epitope spreading, which could generate an abscopal effect beyond the AIM-treated tumor. In some embodiments, AIM therapy is designed to induce an influx of immune cells to transform a “cold” tumor microenvironment to a “hot” tumor microenvironment. In some embodiments, AIM can reinvigorate the host immune system to recognize the tumor as non-self.

In some embodiments, a subject may respond to checkpoint inhibitor immunotherapy, based at least in part on the immune environment of the tumor site. Treating tumors with an allo-immunotherapy can increase the immune response in a tumor environment for a combination therapy (such as AIM and a cancer therapy) to increase response rate and duration of response for subjects suffering from a cancer. This method can be employed in combination with any cancer therapy, for example, those with an immune-activating method of action and could be used in subjects having a cold tumor (e.g., a tumor with limited immune response) or a hot tumor (e.g, a tumor with a high immune response). In some cases, treating a cold tumor with AIM can cause the tumor to become a hot tumor. These methods may provide a therapy for subjects having a cold tumor and may enhance responses for subjects having a hot tumor. Methods as described herein (including AIM), alone or in combination with a cancer therapy may: increase immunogenic tumor cell death, enhance antigen presentation, activate the innate and adaptive immune systems to reverse T-cell exhaustion to combat immunosuppressive cells and factors to increase anti-tumor immunity to eliminate the tumor(s), or any combination thereof. In some cases, administration of an AIM does not cause an anaphylactic response or a cytokine storm in a subject. In some cases, administration of an AIM alone or in conjunction with one or more therapeutics can admonish the activity of a suppressor cell, such as a suppressor B cell and/or a suppressor T cell in a tumor.

In some embodiments, a cancer can be treated using a therapy disclosed herein. In some instances a cancer can be a cancer in an adult, a child, or pediatric subject. In some cases, a cancer may include a lung cancer, an ovarian cancer, a cervical cancer, a uterine cancer, a prostate cancer, a liver cancer, a renal cancer, a pancreatic cancer, a colorectal cancer, a breast cancer, a gastric cancer, a head cancer, a neck cancer, a kidney cancer, a bladder cancer, a brain cancer, a sarcoma cancer, a melanoma, a hematological malignancy, a glioblastoma, or any combination thereof. In some cases, a cancer can comprise a tumor, for example a cancer can comprise a malignant tumor. In some cases, a tumor can comprise comprises a pancreatic tumor, a breast tumor, an ovarian tumor, a prostate tumor, a glioblastoma, or any combination thereof. In some cases, a tumor can comprise a melanoma, a bladder tumor, a head tumor, a neck tumor, a kidney tumor, a liver tumor, a non-small cell lung tumor, or any combination thereof.

In some cases, a lung cancer can comprise a non-small cell lung cancer (NSCLC), a small cell lung cancer (SCLC), or a lung carcinoid tumor. In some embodiments, a cancer can comprise a sarcoma. In some cases, a sarcoma can comprise an osteosarcoma, a dermatofibrosarcoma protuberans, a fibrosarcoma (fibroblastic sarcoma), a chondrosarcoma, a bone cancer, an osteosarcoma, a retroperitoneal a sarcoma, a gynaecological sarcoma, a soft tissue sarcoma, a synovial sarcoma, an Ewing's sarcoma, a rhabdomyosarcoma, a liposarcoma, a synovial sarcoma, a pleomorphic sarcoma, a gastrointestinal stromal tumor, a Kaposi's sarcoma, a leiomyosarcoma, an angiosarcoma, or a combination thereof. In some embodiments, a cancer can comprise a carcinoma. In some cases, a carcinoma can comprise a basal cell carcinoma, a squamous cell carcinoma, a renal cell carcinoma, a ductal carcinoma in situ, an invasive ductal carcinoma, an adenocarcinoma, an adenosquamous carcinoma, an anaplastic carcinoma, a large cell carcinoma, a small cell carcinoma, or a combination thereof. In some cases, a tumor or a cancer can be a liquid tumor or a liquid cancer. In some embodiments, a cancer can comprise a lymphoma or a leukemia. In some cases, a lymphoma can comprise Hodgkin's lymphoma or non-Hodgkin's lymphoma. In some cases, a lymphoma can comprise a diffuse large b-cell lymphoma, an anaplastic large-cell lymphoma, a Burkitt lymphoma, a lymphoblastic lymphoma, a mantle cell lymphoma, a peripheral t-cell lymphoma, a follicular lymphoma, a T-cell lymphoma, a B-cell lymphoma, a cutaneous T-cell lymphoma, a lymphoplasmacytic lymphoma, a marginal zone B-cell lymphoma, a MALT lymphoma, a small-cell a lymphocytic lymphoma, or a combination thereof. In some cases, a leukemia can comprise a lymphocytic leukemia, a myeloid leukemia, an acute leukemia, a chronic leukemia, an acute lymphocytic leukemia (ALL), an acute myelogenous leukemia (AML), a chronic lymphocytic leukemia (CLL), a chronic myelogenous leukemia (CML) or a combination thereof. In some embodiments, a cancer can comprise a germ cell tumor. In some cases, a germ cell tumor can comprise a seminoma or a dysgerminoma. In some cases, a germ cell tumor can comprise an ovarian germ cell tumor, a testicular germ cell tumor, or a extracranial extragonadal germ cell tumor. In some embodiments, a cancer can comprise a blastoma. In some cases, a blastoma can comprise a neuroblastoma, a retinoblastoma, a nephroblastoma, a hepatoblastoma, a medulloblastoma, a pleuropulmonary blastoma, a pancreatoblastoma, or any combination thereof. A cancer may include a pediatric cancer such as a leukemia, a brain tumor, a spinal cord tumor, a neuroblastoma, a Wilms tumor, a retinoblastoma, a bone cancer, a rhabdomyosarcoma, or any combination thereof.

A cancer may comprise a hot tumor, a cold tumor, or a combination thereof. A hot tumor may include a tumor comprising an immune cell rich microenvironment, a high tumor mutational burden, a low number of immunosuppressive cells or factors, expression of a neoantigen, or any combination thereof. In some instances, a neoantigen can be a tumor-specific antigen generated by a mutation in a tumor cell. In some cases, a neoantigen can be recognized by the immune system to generate an immune response. In some cases, a hot tumor can be determined by the number of immune cells in a microenvironment. For example, a hot tumor can have more effector cells than a cold tumor. In some cases, a hot tumor can be determined by the number of immunosuppressive cells in a tumor microenvironment. For example, a hot tumor can have less immunosuppressive cells as compared to a cold tumor. A hot tumor may comprise a melanoma, a bladder cancer, a head cancer, a neck cancer, a kidney cancer, a liver cancer, a lung cancer (such as a non-small cell lung cancer), or any combination thereof. In some cases, a hot tumor can be treated with a checkpoint immunotherapy. A cold tumor may include a tumor comprising a lack of one or more immune cells. In some cases, a cold tumor can include a tumor comprising a presence of an immunosuppressive cell, a presence of an immunosuppressive factor, a lack of response or reduced response to tumor antigen, or any combination thereof. For example, a cold tumor can have an increased number of immunosuppressive cells as compared to a hot tumor. In some instances, a checkpoint immunotherapy may not be effective for treating a cold tumor. A cold tumor may comprise a glioblastoma, a pancreatic cancer, a breast cancer, a prostate cancer, an ovarian cancer, or any combination thereof. In some cases, a cold tumor can be determined by the number of immune cells in a microenvironment. For example, a cold tumor can have a decreased number of immune effector cells (e.g. cells) as compared to a hot tumor. In some cases, a cold tumor can be determined by the number of immunosuppressive cells in a tumor microenvironment. For example, a cold tumor can have an increased number of T-regulatory cells (T_{reg}) or other immune effector cells (e.g., cancer-associated fibroblasts, myeloid-derived suppressor cells, and tumor-associated macrophages) as compared to a hot tumor.

In some embodiments, a subject can be diagnosed with a disease. In some cases, a subject can be diagnosed with a disease prior to a treatment. In some cases, a subject can be diagnosed with a disease during a treatment. In some cases, a diagnostic test can be performed on an sample from a subject. In some instances, a diagnostic test for a disease or condition can be performed. In some cases, a diagnostic test can be an in vivo or an in vitro test. In some cases, a diagnostic test can comprise a polymerase chain reaction (PCR) test, a sequencing test (e.g., next generation sequencing assay), a serological assay, an enzyme-linked immunoassay (ELISA), or a combination thereof. In some cases, a diagnostic test can be an imaging assay, such as a radiological imaging assay. In some cases, a radiological imaging assay can comprise a computed tomography (CT), a magnetic resonance imaging (MRI), a magnetic resonance angiography, a mammography, a bone scan, a thyroid scan, a X-ray, a positron emission tomography (PET) scan, an ultrasound, or any combination thereof.

### Administration and Dosing

The terms “administer,” “administering”, “administration,” and the like, as used herein, refers to methods that are used to enable delivery of compositions disclosed herein to the desired site of biological action. In some cases, a composition herein can be a pharmaceutical composition. Delivery can include direct application to the affected tissue or region of the body. Delivery can include proximal application to the affected tissue or region of the body. In some cases, delivery can include proximal and direct application to the affected tissue or region of the body. A composition provided herein can be administered by any method. A method of administration can be by a cell transplant, a tissue transplant, an HLA molecule transplant, or a combination thereof. In some cases, a composition can be delivered by parenchymal injection, intra-thecal injection, intra-ventricular injection, intra-tumoral injection, intra-cisternal injection, or any combination thereof. In some cases, a method of administration can be by inhalation, intraarterial injection, intracerebroventricular injection, intracisternal injection, intramuscular injection, intraorbital injection, intraparenchymal injection, intraperitoneal injection, intraspinal injection, intrathecal injection, intravenous injection, intraventricular injection, stereotactic injection, subcutaneous injection, or any combination thereof. Delivery can include parenteral administration (including intravenous, subcutaneous, intrathecal, intraperitoneal, intramuscular, intravascular or infusion), oral administration, nasal administration, inhalation administration, vaginal administration, penile administration, anal administration, intraduodenal administration, rectal administration. In some cases, delivery can include delivery of a composition by a surgery, or by an injection. In some cases, delivery can include delivery of a composition by a device, wherein the device is configured to implant a composition to a specific area. Delivery can include topical administration (such as a lotion, a cream, a gel, a liquid, a solid, a powder, an ointment) to an external surface of a surface, such as a skin. In some instances, a subject can administer the composition in the absence of supervision. In some instances, a subject can administer a composition under the supervision of a medical professional (e.g., a physician, nurse, physician's assistant, orderly, hospice worker, etc.). In some cases, a medical professional administers a composition disclosed herein.

In some cases, a vector can be employed to deliver a composition disclosed herein. In some embodiments, a vector can comprise a cell, an HLA molecule or a fragment thereof, an antigenic marker or any combination thereof. In some cases, a vector can be a natural vector or a synthetic vector. In some cases, a vector can be a vector that can be modified from a naturally occurring vector. Any vector can be utilized. In some cases, a vector can comprise a nanoparticle, a viral vector, a viral-like particle, a liposome, an exosome, an extracellular vesicle, or a combination thereof. For example, an HLA molecule or a fragment thereof can be incorporated into the envelope of a viral vector on onto the surface of a modified liposome. In some cases, a vector can comprise an allogeneic cell/tissue, a xenogeneic cell/tissue, such as an adipose cell.

In some embodiments, a composition herein can be in the form of a dose. In some cases, dose can be administered to a subject in need thereof. In some cases, a dose can be in unit dose form. In some instances, a dosage form can comprise an aerosol, a bar, a food, a bead, a capsule, a sheet, a gel, a concentrate, a cream, a dressing, an enema, a film, a solution, a suspension, a gel, a granule, a powder, an implant, an injection, a jelly, a liquid, a lotion, a lozenge, a paste, a patch, a pill, a powder, a spray, a suppository, a syrup, a tablet, a tincture, a troche, or a combination thereof. In some cases, a dosage can be in the form of a liquid. In some embodiments, a dosage can be in the form of an injection. In some cases, a composition herein can be a lyophilized composition. In some cases, a composition herein can comprise an adjuvant. In some cases, a composition herein can comprise a vaccine.

In some embodiments, an allogeneic cell or a xenogeneic cell can be administered as a composition to a subject in need thereof. In some cases, an allogeneic cell or a xenogeneic cell be administered to a subject in a dose of equal to about, or more than about: 1×10¹ cells, 1×10² cells, 1×10³ cells, 1×10⁴ cells, 1×10⁵ cells, 1×10⁶ cells, 1×10⁷ cells, 1×10⁸ cells, 1×10⁹ cells, 1×10¹⁰ or 1×10¹¹ cells. In some cases, an allogeneic cell or a xenogeneic cell can be administered to a subject in a dose of less than about: 1*10¹ cells, 1×10² cells, 1×10³ cells, 1×10⁴ cells, 1×10⁵ cells, 1×10⁶ cells, 1×10⁷ cells, 1×10⁸ cells, 1×10⁹ cells, 1×10¹⁰ or 1×10¹¹ cells. In some cases, an allogeneic cell or a xenogeneic cell can be administered to a subject in a dose of about: 5×10¹ cells to about 5×10² cells, 5×10² cells to about 5×10⁴ cells, 5×10⁴ cells to about 5×10⁵ cells, 5×10⁵ cells to about 5×10⁶ cells, 5×10⁶ cells to about 5×10⁷ cells, 5×10⁷ cells to about 5×10⁸ cells, 5×10⁸ cells to about 5×10⁹ cells, 5×10⁹ cells to about 5×10¹⁰ cells, or about 5×10¹⁰ cells to about 5×10¹¹ cells.

In some cases, a dose of a composition described herein can be administered in unit dose form in amounts from about 0.001 milligram (mg) to about 10000 mg. In some cases, a dose of a composition herein can be administered in amounts from about: 0.001 mg to about 0.01 mg, 0.01 mg to about 0.1 mg, 0.1 mg to about 1 mg, 1 mg to about 10 mg, 10 mg to about 100 mg, 100 mg to about 1000 mg, or 1000 mg to about 10000 mg. In some cases, a subject can be dosed with a composition in an amount ranging from about 0.001 mg/kilogram (kg) of body weight of the subject to about 100 g/kg of body weight of the subject. In some cases, the dosage can be based on a mg of a composition, per kg of subject body weight, and can be more than about, less than about, or equal to about: 0.0001, 0.001, 0.01. 0.1, 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, 1000, or 10000 mg/kg of subject body weight. In some cases, a dose of a composition described herein can be administered in unit dose form in amounts from about 0.001 cubic centimeter (cc) to about 1000 cc. In some cases, a dose of a composition herein can be administered in amounts from about: 0.001 cc to about 0.01 cc, 0.01 cc to about 0.1 cc, 0.1 cc to about 1 cc, 1 cc to about 10 cc, 10 cc to about 100 cc, or 100 cc to about 1000 cc. In some cases, a dose of a composition herein can be administered in amounts of more than about, less than about, or equal to about: 0.001 cc, 0.01 cc, 0.1 cc, 0.2 cc, 0.3 cc, 0.4 cc, 0.5 cc, 0.6 cc, 0.7 cc, 0.8 cc, 0.9 cc, 1 cc, 2 cc, 3 cc, 4 cc, 5 cc, 6 cc, 7 cc, 8 cc, 9 cc, 10 cc, 11 cc, 12 cc, 13 cc, 14 cc, 15 cc, 16 cc, 17 cc, 18 cc, 19 cc, 20 cc, 21 cc, 22 cc, 23 cc, 24 cc, 25 cc, 26 cc, 27 cc, 28 cc, 29 cc, 30 cc, 31 cc, 32 cc, 33 cc, 34 cc, 35 cc, 36 cc, 37 cc, 38 cc, 39 cc, 40 cc, 41 cc, 42 cc, 43 cc, 44 cc, 45 cc, 46 cc, 47 cc, 48 cc, 49 cc, 50 cc, 55 cc, 60 cc, 65 cc, 70 cc, 75 cc, 80 cc, 85 cc, 90 cc, 95 cc, 100 cc, 105 cc, 110 cc, 115 cc, 120 cc, 125 cc, 130 cc, 135 cc, 140 cc, 145 cc, 150 cc, 155 cc, 160 cc, 165 cc, 170 cc, 175 cc, 180 cc, 185 cc, 190 cc, 195 cc, 200 cc, 205 cc, 210 cc, 215 cc, 220 cc, 225 cc, 230 cc, 235 cc, 240 cc, 245 cc, 250 cc, 255 cc, 260 cc, 265 cc, 270 cc, 275 cc, 280 cc, 285 cc, 290 cc, 295 cc, 300 cc, 305 cc, 310 cc, 315 cc, 320 cc, 325 cc, 330 cc, 335 cc, 340 cc, 345 cc, 350 cc, 355 cc, 360 cc, 365 cc, 370 cc, 375 cc, 380 cc, 385 cc, 390 cc, 395 cc, 400 cc, 405 cc, 410 cc, 415 cc, 420 cc, 425 cc, 430 cc, 435 cc, 440 cc, 445 cc, 450 cc, 455 cc, 460 cc, 465 cc, 470 cc, 475 cc, 480 cc, 485 cc, 490 cc, 495 cc, 500 cc, 600 cc, 700 cc, 800 cc, 900 cc, 1000 cc, 1100 cc, 1200 cc, 1300 cc, 1400 cc, 1500 cc, 1600 cc, 1700 cc, 1800 cc, 1900 cc, or 2000 cc.

In some embodiments, administering can be performed at least about: 1 time per day, 2 times per day, 3 times per day, 4 times per day, 5 times per day, 6 times per day, 7 times per day, or more than 7 times per day. In some cases, administering can be performed daily, weekly, monthly, or as needed. In some cases, administration or application of a composition or a therapy disclosed herein can be performed continuously throughout a 24 hour period, for example, when an implant can be used for administration.

In some cases, administration or application of a composition or a therapy disclosed herein can be performed at least 1, 2, 3, 4, 5, 6, 7, 8, 9, 10, 11, 12, 13, 14, 15, 16, 17, 18, 19, 20, or 21 times a week. In some cases, administration or application of composition or a therapy disclosed herein can be performed at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month. In some cases, a composition can be administered as a single dose or as divided doses. In some cases, a composition or a therapy described herein can be administered at a first time point and a second time point. In some cases, a composition or a therapy can be administered such that a first administration can be administered before the other with a difference in administration time of 1 hour, 2 hours, 4 hours, 8 hours, 12 hours, 16 hours, 20 hours, 1 day, 2 days, 4 days, 7 days, 2 weeks, 4 weeks, 2 months, 3 months, 4 months, 5 months, 6 months, 7 months, 8 months, 9 months, 10 months, 11 months, 1 year or more.

Administration or application of a composition or a therapy disclosed herein can be performed for a treatment duration of at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 550, 600, 650, 700, 750, 800, 850, 900, 950 or 1000 days consecutive or nonconsecutive days. In some cases, a treatment duration can be from about: 1 to about 30 days, 1 to about 60 days, 1 to about 90 days, 30 days to about 90 days, 60 days to about 90 days, 30 days to about 180 days, from 90 days to about 180 days, or from 180 days to about 360 days. In some cases, a treatment duration can until the tumor is no longer present or identifiable by a diagnostic device.

Administration or application of a composition or a therapy disclosed herein can be performed for a treatment duration of at least about 1 week, at least about 2 weeks, at least about 3 weeks, at least about 4 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 1 year, at least about 2 years, at least about 3 years, at least about 4 years, at least about 5 years, at least about 6 years, at least about 7 years, at least about 8 years, at least about 9 years, at least about 10 years, at least about 15 years, at least about 20 years, or for life. Administration can be performed repeatedly over a lifetime of a subject, such as once a month or once a year for the lifetime of a subject. Administration can be performed repeatedly over a substantial portion of a subject's life, such as once a month or once a year for at least about 1 year, 5 years, 10 years, 15 years, 20 years, 25 years, 30 years, or more.

In some embodiments, a composition or a therapy can be administered as needed, or for: one day, two days, three days, four days, five days, six days, a week, two weeks, three weeks, a month, two months, three months, four months, five months, six months, seven months, eight months, nine months, ten months, eleven months, a year, or chronically. In some embodiments, administering can be performed for about: 1 day to about 8 days, 1 week to about 5 weeks, 1 month to about 12 months, 1 year to about 3 years, 3 years to about 10 years, 10 years to about 50 years, 25 years to about 100 years, or 50 years to about 130 years.

In some embodiments, a composition can be administered with one or more additional cancer therapies. For example, a composition disclosed herein can be administered with 1, 2, 3, 4, 5, 6, 8, 8, 9 or more additional cancer therapies. In some cases, a composition herein can be administered with two cancer therapies. In some cases, a composition (e.g., an allogeneic cell, or an HLA molecule or a portion thereof) can be administered concurrently with one or more additional cancer therapies. In some cases, a composition (e.g., an allogeneic cell, or an HLA molecule or a portion thereof) can be administered consecutively with one or more additional cancer therapies.

In some embodiments, a cancer therapy can comprise an adoptive cell therapy, an immunotherapy, a chemotherapy, a radiation therapy, a surgery, a hormone therapy, a bone marrow transplant, or any combination thereof. In some embodiments, a cancer therapy can comprise a monoclonal antibody, a small molecule, or any combination thereof. In some cases, an adoptive cell therapy can comprise chimeric antigen receptor (CAR) immune cell therapy, an engineered T-cell receptor (TCR) therapy, a tumor-infiltrating lymphocyte (TIL) therapy, or an NK cell therapy. In some cases, a CAR immune cell therapy can comprise a CAR-T cell therapy, a CAR-NK cell therapy, a CAR-B cell therapy, a CAR macrophage, a CAR induced pluripotent stem cell (IPSC), a CAR stem cell (e.g., an embryonic stem cell), or a combination thereof. In some cases, a IPSC or stem cell can be sourced from blood, cord blood, bone marrow, or any combination thereof. In some cases, a CAR immune cell therapy can be configured to target a biomarker of a cancer cell. In some cases, a biomarker of a cancer cell can comprise CD30, CD20, CD19, CD22, CD138, CD44v6, CAIX, CEA, CD133, c-Met, EGFR, EGFRvIII, Epcam, EphA2, fetal acetylcholine receptor, FR alpha, GD2, GPC3, GUCY2C, HER1, HER2, ICAM-1, IL13R alpha 2, IL11R alpha, Kras, Kras G12D, L1CAM, MAGE, MET, mesothelin, MUC1, MUC16, NKG2D, NY-ESO-1, PSCA, WT-1, or any combination thereof. In some cases, an immunotherapy can comprise a checkpoint inhibitor. In some cases, an immunotherapy can comprise a PD-1 inhibitor (e.g., pembrolizumab, nivolumab, cemiplimab, a salt of any of these, or a biosimilar of any of these), a PD-L1 inhibitor (e.g., atezolizumab, avelumab, durvalumab a salt of any of these, or a biosimilar of any of these), a CTLA-4 inhibitor (e.g., ipilimumab, a salt thereof, or a biosimilar of any of these), a TIGIT inhibitor, a TF inhibitor (e.g., tisotumab vedotin-tftv, a salt thereof, or a biosimilar of any of these), a TIGIT inhibitor, a ATAR inhibitor, a B7-H3 inhibitor, a B7-H4 inhibitor, a BTLA inhibitor, an IDO inhibitor, a KIR inhibitor, a LAG inhibitor, a NOX2 inhibitor, a SIGLEC7 inhibitor, a SIGLEC9 inhibitor, a TIM3 inhibitor, a VISTA inhibitor, or any combination thereof. In some cases, an immunotherapy can comprise a monoclonal antibody, a bispecific antibody, a conjugated antibody (e.g., an antibody drug conjugant), a oncolytic virus therapy (e.g., talimogene laherparepvec, a salt thereof, or a biosimilar of any of these), a cancer vaccine (e.g., cervarix, Gardasil, Gardasil-9, HEPLISAV-B, sipuleucel-T, Bacillus Calmette-Guerin (BCG). In some cases, an immunotherapy can comprise an immune system modulator. In some cases, an immune system modulator can comprise an interleukin (e.g., IL-2), an interleukin inhibitor (e.g., an IL-6 inhibitor), an antihistamine, an immunomodulator (e.g., imiquimod, lenalidomide, pomalidomide, thalidomide, a salt thereof, or a derivative thereof), or any combination thereof. In some cases, a chemotherapy can comprise altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, carmustine, lomustine, streptozocin, azacitidine, 5-fluorouracil (5-fu), 6-mercaptopurine (6-mp), capecitabine (xeloda), cladribine, clofarabine, cytarabine (ara-c), decitabine, floxuridine, fludarabine, gemcitabine (gemzar), hydroxyurea, methotrexate, nelarabine, pemetrexed, pentostatin, pralatrexate, thioguanine, trifluridine/tipiracil, daunorubicin, doxorubicin (adriamycin), doxorubicin liposomal, epirubicin, idarubicin, valrubicin, bleomycin, dactinomycin, mitomycin-c, mitoxantrone, irinotecan, irinotecan liposomal, topotecan, etoposide (vp-16), mitoxantrone (also acts as an anti-tumor antibiotic), teniposide, cabazitaxel, docetaxel, nab-paclitaxel, paclitaxel, vinblastine, vincristine, vincristine liposomal, vinorelbine, prednisone, methylprednisolone, dexamethasone, all-trans-retinoic acid, arsenic trioxide, asparaginase, eribulin, hydroxyurea, ixabepilone, mitotane, omacetaxine, pegaspargase, procarbazine, romidepsin, vorinostat, a salt of any of these, or any combination thereof. In some cases, a radiation therapy can comprise a 3D conformal radiation therapy, an image guided radiation therapy (IGRT), a intensity modulated radiation therapy (IMRT), a volumetric modulated arc therapy, an internal radiation therapy, a brachytherapy, an intraoperative radiation therapy, a stereotactic radiosurgery, an external beam radiation therapy, a proton therapy, a stereotactic body radiation therapy, or any combination thereof. In some cases, a hormone therapy can comprise an anti-androgens, such as apalutamide, enzalutamide, darolutamide, bicalutamide, flutamide, nilutamide, or a salt of any of these. In some cases, a hormone therapy can comprise a CYP17 inhibitor or a luteinizing hormone-releasing hormone (LHRH) agonists and antagonists. In some cases, a hormone therapy can comprise abiraterone, ketoconazole, goserelin, leuprolide, triptorelin, degarelix, a salt of any of these, or any combination thereof. In some cases, a hormone therapy can comprise medroxyprogesterone, megestrol, tamoxifen, raloxifene, goserelin, leuprolide, letrozole, anastrozole, exemestane, mitotane, fulvestrant, toremifene, a salt of any of these, or any combination thereof. In some cases, a bone marrow transplant can comprise an autologous bone marrow transplant, an allogeneic bone marrow transplant, or an umbilical cord blood transplant.

In some embodiments, a cancer therapy can comprise abemaciclib, abemaciclib, abiraterone acetate, abiraterone acetate, acalabrutinib, ado-trastuzumab emtansine, afatinib dimaleate, aldesleukin, alectinib, alemtuzumab, alpelisib, amifostine, aminolevulinic acid hydrochloride, anastrozole, apalutamide, aprepitant, arsenic trioxide, atezolizumab, avelumab, axicabtagene ciloleucel, axitinib, azacytidine, belinostat, bendamustine hydrochloride, bevacizumab, bexarotene, bicalutamide, binimetinib, bleomycin sulfate, blinatumomab, bortezomib, bosutinib, brentuximab vedotin, brigatinib, busulfan, cabazitaxel, cabozantinib-s-malate, calaspargase pegol-mknl, capecitabine, caplacizumab-yhdp, carboplatin, carfilzomib, carmustine, cemiplimab-rwlc, ceritinib, cetuximab, chlorambucil, cisplatin, cladribine, clofarabine, cobimetinib, copanlisib hydrochloride, crizotinib, cyclophosphamide, cytarabine, dabrafenib mesylate, dacarbazine, dacomitinib, dactinomycin, daratumumab, darbepoetin alfa, darolutamide, dasatinib, daunorubicin hydrochloride, decitabine, defibrotide sodium, degarelix, denileukin diftitox, denosumab, dexamethasone, dexrazoxane hydrochloride, dinutuximab, docetaxel, doxorubicin hydrochloride, durvalumab, duvelisib, elotuzumab, eltrombopag olamine, emapalumab-lzsg, enasidenib mesylate, encorafenib, entrectinib, enzalutamide, epirubicin hydrochloride, epoetin alfa, erdafitinib, eribulin mesylate, erlotinib hydrochloride, etoposide, everolimus, exemestane, fam-trastuzumab deruxtecan-nxki, fedratinib hydrochloride, filgrastim, fludarabine phosphate, fluorouracil, flutamide, fostamatinib disodium, fulvestrant, gefitinib, gemcitabine hydrochloride, gemcitabine-cisplatin, gemcitabine-oxaliplatin, gemtuzumab ozogamicin, gilteritinib fumarate, glasdegib maleate, glucarpidase, goserelin acetate, granisetron, granisetron hydrochloride, hydroxyurea, ibritumomab tiuxetan, ibrutinib, idarubicin hydrochloride, idelalisib, ifosfamide, imatinib mesylate, imiquimod, inotuzumab ozogamicin, iobenguane i131, ipilimumab, irinotecan hydrochloride, ivosidenib, ixabepilone, ixazomib citrate, lanreotide acetate, lapatinib ditosylate, larotrectinib sulfate, lenalidomide, lenvatinib mesylate, letrozole, leucovorin calcium, leuprolide acetate, lomustine, lorlatinib, lu 177-dotatate, mechlorethamine hydrochloride, megestrol acetate, melphalan, melphalan hydrochloride, mercaptopurine, methotrexate, methylnaltrexone bromide, midostaurin, mitomycin c, mitoxantrone hydrochloride, mogamulizumab-kpkc, moxetumomab pasudotox-tdfk, necitumumab, nelarabine, neratinib maleate, nilotinib, nilutamide, niraparib tosylate monohydrate, nivolumab, obinutuzumab, ofatumumab, olaparib, omacetaxine mepesuccinate, ondansetron hydrochloride, osimertinib mesylate, oxaliplatin, paclitaxel, palbociclib, palifermin, palonosetron hydrochloride, pamidronate disodium, panitumumab, panobinostat, pazopanib hydrochloride, pegaspargase, pegfilgrastim, peginterferon alfa-2b, pembrolizumab, pemetrexed disodium, pertuzumab, plerixafor, polatuzumab vedotin-piiq, pomalidomide, ponatinib hydrochloride, pralatrexate, prednisone, procarbazine hydrochloride, propranolol hydrochloride, radium 223 dichloride, raloxifene hydrochloride, ramucirumab, rasburicase, ravulizumab-cwvz, recombinant interferon alfa-2b, regorafenib, ribociclib, rituximab, rolapitant hydrochloride, romidepsin, romiplostim, rucaparib camsylate, ruxolitinib phosphate, selinexor, siltuximab, sipuleucel-t, sonidegib, sorafenib tosylate, sunitinib malate, tagraxofusp-erzs, talazoparib tosylate, talc, talimogene laherparepvec, tamoxifen citrate, temozolomide, temsirolimus, thalidomide, thioguanine, thiotepa, tisagenlecleucel, tocilizumab, topotecan hydrochloride, toremifene, trabectedin, trametinib, trastuzumab uridine, triacetate, valrubicin, vandetanib, vemurafenib, venetoclax, vincristine sulfate, vinorelbine tartrate, vismodegib, vorinostat, zanubrutinib, ziv-aflibercept, zoledronic acid, a pharmaceutically acceptable salt of any of these, a biological active fragment of any of these, a biosimilar of any of these, or any combination thereof.

In some embodiments, a cancer therapy can comprise a drug that is licensed or approved for the treatment of cancer by a regulatory agency. For example, a drug can be approved for the treatment of cancer by the Food and Drug Administration (FDA) or the European Medicines Agency (EMA).

### Kits

Kits and articles of manufacture are also described for use of the compositions described herein. In some embodiments, a kit can comprise an allogeneic cell and/or a xenogeneic cell and instructions for use in administering the allogeneic cell and/or the xenogeneic cell to the subject to treat a cancer. In some embodiments, such kits can include a carrier, package, and/or container. In some embodiments, the container can contain capsules, vials, or tubes. In some cases, a container can be a plastic, a metal, a glass, or any suitable material. In some embodiments, the kit can be accompanied by instructions for administration. In some embodiments, a kit can be in a packaging.

In some embodiments, packaging can also be accompanied with a notice as required by a governmental agency regulating the manufacture, use, or sale of pharmaceuticals. This notice can state that the drug is approved by the agency for human or veterinary administration. Such notice, for example, is the labeling approved by the U.S. Food and Drug Administration for prescription drugs, or the approved product insert. In some cases, compositions provided herein when formulated with a compatible excipient, diluent and/or carrier can also be prepared, placed in an appropriate container, and labeled for treatment of an indicated condition.

Such kits can comprise an identifying description or label for the containers. In some embodiments, the label can be on a container with letters, numbers or other characters forming the label and attached, molded or etched into the container itself. In some cases, a label can be associated with a container when it is present within a receptacle or carrier that also holds the container, e.g., as a package insert. In some embodiments, a label can be used to indicate that the contents are to be used for a specific therapeutic application. In yet other embodiments, the label can indicate directions for use of the contents, such as in the methods described herein. In some embodiments, a set of instructions may also be included, generally in the form of a package insert. The informational material may contain instructions on how to dispense the pharmaceutical composition, including description of the type of patients who may be treated, the schedule (e.g., dose and frequency), and the like.

### Numbered Embodiments

A number of compositions, and methods are disclosed herein. Specific exemplary embodiments of these compositions and methods are disclosed below. The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

Embodiment 1. A method of treating a subject having a cancer, the method comprising: administering a therapeutically effective amount of an allogeneic cell to the subject, wherein an antigen marker profile of a cell of the subject comprises at least one mismatched antigen marker as compared to an antigen marker profile of the allogeneic cell administered to the subject.

Embodiment 2. The method of embodiment 1, wherein the cancer comprises a malignant tumor.

Embodiment 3. The method of embodiment 1, wherein the antigen marker profile comprises a human leukocyte antigen (HLA) or a portion thereof.

Embodiment 4. The method of embodiment 1, wherein the at least one mismatched antigen marker is 2 mismatched antigen markers.

Embodiment 5. The method of embodiment 1, wherein the at least one mismatched antigen marker is 3 mismatched antigen markers.

Embodiment 6 The method of embodiment 1, wherein a cell transplant or tissue transplant comprising the allogeneic cell is administered to the subject.

Embodiment 7. The method of embodiment 1, further comprising: administering an isolated and purified HLA or a portion thereof to the subject, wherein the isolated and purified HLA or the portion thereof is mismatched to an antigen marker profile of a cell of the subject.

Embodiment 8. The method of embodiment 7, wherein the HLA or portion thereof is a recombinant HLA or recombinant portion thereof.

Embodiment 9. The method of embodiment 1, further comprising, administering a cancer therapy to the subject.

Embodiment 10. The method of embodiment 9, wherein the allogeneic cell and the cancer therapy are administered concurrently.

Embodiment 11. The method of embodiment 9, wherein the allogeneic cell and the cancer therapy are administered consecutively.

Embodiment 12. The method of embodiment 9, wherein the cancer therapy comprises at least two cancer therapies.

Embodiment 13. The method of embodiment 9, wherein the cancer therapy comprises a chimeric antigen receptor (CAR) T cell therapy, an immunotherapy, a chemotherapy, a radiation therapy, a surgery, a hormone therapy, a bone marrow transplant, or any combination thereof.

Embodiment 14. The method of embodiment 13, wherein the cancer therapy comprises the CAR-T cell therapy, and wherein the CAR-T therapy is configured to target a biomarker of a cancer cell, wherein the biomarker comprises CD30, CD20 CD19, CD22, CD138, or any combination thereof.

Embodiment 15. The method of embodiment 13, wherein the cancer therapy comprises the immunotherapy.

Embodiment 16 The method of embodiment 15, wherein the immunotherapy comprises a checkpoint inhibitor.

Embodiment 17. The method of embodiment 16, wherein the checkpoint inhibitor comprises a CTLA-4 inhibitor, a PD-1 inhibitor, a PD-L1 inhibitor, a biosimilar of any of these, or any combination thereof.

Embodiment 18. The method of embodiment 16, wherein the checkpoint inhibitor comprises pembrolizumab, nivolumab, ipilimumab, atezolizumab, avelumab, durvalumab, a salt of any of these, a biosimilar of any of these, or any combination thereof.

Embodiment 19. The method of embodiment 9, wherein the cancer therapy comprises a drug that is licensed and approved for treatment of the cancer by a regulatory agency.

Embodiment 20. The method of embodiment 1, wherein the cancer comprises a glioblastoma, a pancreatic cancer, a breast cancer, a prostate cancer, an ovarian cancer, or any combination thereof.

Embodiment 21. The method of embodiment 1, wherein the cancer comprises a melanoma, a bladder cancer, a head or neck cancer, a kidney cancer, a liver cancer, a non-small cell lung cancer, or any combination thereof.

Embodiment 22. The method of embodiment 2, wherein the malignant tumor comprises a pancreatic tumor, a breast tumor, an ovarian tumor, a prostate tumor, a glioblastoma, or any combination thereof.

Embodiment 23. The method of embodiment 2, wherein the malignant tumor comprises a melanoma, a bladder tumor, a head tumor, a neck tumor, a kidney tumor, a liver tumor, a non-small cell lung tumor, or any combination thereof.

Embodiment 24. The method of embodiment 2, wherein the allogeneic cell is administered to or proximal to the malignant tumor.

Embodiment 25. The method of embodiment 1, wherein the administering of the allogeneic cell is by parenchymal injection, intra-thecal injection, intra-ventricular injection, intra-cisternal injection, or any combination thereof.

Embodiment 26. The method of embodiment 1, wherein the administering of the allogeneic cell occurs from about once a day to about once a year.

Embodiment 27. The method of embodiment 1, wherein the administering of the allogeneic cell reduces a size of a tumor in the subject.

Embodiment 28. The method of embodiment 1, the administering of the allogeneic cell inhibits growth of a tumor in the subject.

Embodiment 29. The method of embodiment 1, wherein the allogeneic cell is an isolated and purified allogeneic cell.

Embodiment 30. The method of embodiment 1, wherein the allogeneic cell does not comprise a hematopoietic cell.

Embodiment 31. The method of embodiment 1, wherein the allogeneic cell comprises a CAR T cell.

Embodiment 32. The method of embodiment 1, wherein the allogeneic cell comprises a B cell, a T cell, an NK cell, a macrophage, a dendritic cell, an endothelial cell, a stem cell, or any combination thereof.

Embodiment 33. The method of embodiment 1, wherein the allogeneic cell comprises an immune cell, a bone cell, a fat cell, a nerve cell, a skin cell, a pancreatic cell, a sex cell, a blood cell, a muscle cell, a stem cell or any combination thereof.

Embodiment 34. The method of embodiment 1, wherein a tissue that comprises the allogeneic cell is administered to the subject.

Embodiment 35. The method of embodiment 1, further comprising performing an analysis on a sample comprising the allogeneic cell.

Embodiment 36 The method of embodiment 35, wherein the analysis comprises: performing a genetic assay on at least a portion of the sample; performing an epigenetic assay on at least a portion of the sample; obtaining at least a portion of a medical history of a donor of the sample; identifying a presence or absence of one or more cancer mutations in genetic material obtained from at least a portion of the sample, or any combination thereof.

Embodiment 37. The method of embodiment 1, further comprising selecting the allogeneic cell from a plurality of allogeneic cells.

Embodiment 38. A cell bank comprising the allogeneic cell of embodiment 1.

Embodiment 39. The method of embodiment 1, wherein the subject is a human subject.

Embodiment 40. The method of embodiment 1, wherein a sample obtained from the subject has been evaluated by a diagnostic test.

Embodiment 41. The method of embodiment 40, wherein the diagnostic test is an FDA approved or FDA cleared diagnostic test.

Embodiment 42. A kit comprising the allogeneic cell of embodiment 1 and instructions for use in administering the allogeneic cell to the subject to treat the cancer.

Embodiment 43. A pharmaceutical composition comprising the allogeneic cell of embodiment 1 in unit dose form and a pharmaceutically acceptable excipient, carrier, or diluent.

Embodiment 44. The pharmaceutical composition of embodiment 43, wherein the pharmaceutical composition is encapsulated.

Embodiment 45. The pharmaceutical composition of embodiment 43, wherein the pharmaceutical composition is in the form of an injectable liquid.

Embodiment 46 A method of treating a subject having a cancer, the method comprising: administering a therapeutically effective amount of an isolated and purified HLA or a portion thereof to the subject, wherein the isolated and purified HLA or the portion thereof is mismatched to an antigen marker profile of a cell of the subject.

Embodiment 47. The method of embodiment 46, wherein the HLA or portion thereof is a synthetic HLA or synthetic portion thereof.

Embodiment 48. The method of embodiment 46, wherein the HLA or portion thereof is a recombinant HLA or recombinant portion thereof.

Embodiment 49. The method of embodiment 46, wherein more than one isolated and purified HLA or portions thereof are administered to the subject.

Embodiment 50. The method of embodiment 46, further comprising administering an allogeneic cell to the subject, wherein an antigen marker profile of a cell of the subject comprises at least one mismatched antigen marker as compared to an antigen marker profile of the allogeneic cell administered to the subject.

Embodiment 51. The method of embodiment 9, wherein the cancer therapy comprises: abemaciclib, abemaciclib, abiraterone acetate, abiraterone acetate, acalabrutinib, ado-trastuzumab emtansine, afatinib dimaleate, aldesleukin, alectinib, alemtuzumab, alpelisib, amifostine, aminolevulinic acid hydrochloride, anastrozole, apalutamide, aprepitant, arsenic trioxide, atezolizumab, avelumab, axicabtagene ciloleucel, axitinib, azacytidine, belinostat, bendamustine hydrochloride, bevacizumab, bexarotene, bicalutamide, binimetinib, bleomycin sulfate, blinatumomab, bortezomib, bosutinib, brentuximab vedotin, brigatinib, busulfan, cabazitaxel, cabozantinib-s-malate, calaspargase pegol-mknl, capecitabine, caplacizumab-yhdp, carboplatin, carfilzomib, carmustine, cemiplimab-rwlc, ceritinib, cetuximab, chlorambucil, cisplatin, cladribine, clofarabine, cobimetinib, copanlisib hydrochloride, crizotinib, cyclophosphamide, cytarabine, dabrafenib mesylate, dacarbazine, dacomitinib, dactinomycin, daratumumab, darbepoetin alfa, darolutamide, dasatinib, daunorubicin hydrochloride, decitabine, defibrotide sodium, degarelix, denileukin diftitox, denosumab, dexamethasone, dexrazoxane hydrochloride, dinutuximab, docetaxel, doxorubicin hydrochloride, durvalumab, duvelisib, elotuzumab, eltrombopag olamine, emapalumab-lzsg, enasidenib mesylate, encorafenib, entrectinib, enzalutamide, epirubicin hydrochloride, epoetin alfa, erdafitinib, eribulin mesylate, erlotinib hydrochloride, etoposide, everolimus, exemestane, fam-trastuzumab deruxtecan-nxki, fedratinib hydrochloride, filgrastim, fludarabine phosphate, fluorouracil, flutamide, fostamatinib disodium, fulvestrant, gefitinib, gemcitabine hydrochloride, gemcitabine-cisplatin, gemcitabine-oxaliplatin, gemtuzumab ozogamicin, gilteritinib fumarate, glasdegib maleate, glucarpidase, goserelin acetate, granisetron, granisetron hydrochloride, hydroxyurea, ibritumomab tiuxetan, ibrutinib, idarubicin hydrochloride, idelalisib, ifosfamide, imatinib mesylate, imiquimod, inotuzumab ozogamicin, iobenguane i131, ipilimumab, irinotecan hydrochloride, ivosidenib, ixabepilone, ixazomib citrate, lanreotide acetate, lapatinib ditosylate, larotrectinib sulfate, lenalidomide, lenvatinib mesylate, letrozole, leucovorin calcium, leuprolide acetate, lomustine, lorlatinib, lu 177-dotatate, mechlorethamine hydrochloride, megestrol acetate, melphalan, melphalan hydrochloride, mercaptopurine, methotrexate, methylnaltrexone bromide, midostaurin, mitomycin c, mitoxantrone hydrochloride, mogamulizumab-kpkc, moxetumomab pasudotox-tdfk, necitumumab, nelarabine, neratinib maleate, nilotinib, nilutamide, niraparib tosylate monohydrate, nivolumab, obinutuzumab, ofatumumab, olaparib, omacetaxine mepesuccinate, ondansetron hydrochloride, osimertinib mesylate, oxaliplatin, paclitaxel, palbociclib, palifermin, palonosetron hydrochloride, pamidronate disodium, panitumumab, panobinostat, pazopanib hydrochloride, pegaspargase, pegfilgrastim, peginterferon alfa-2b, pembrolizumab, pemetrexed disodium, pertuzumab, plerixafor, polatuzumab vedotin-piiq, pomalidomide, ponatinib hydrochloride, pralatrexate, prednisone, procarbazine hydrochloride, propranolol hydrochloride, radium 223 dichloride, raloxifene hydrochloride, ramucirumab, rasburicase, ravulizumab-cwvz, recombinant interferon alfa-2b, regorafenib, ribociclib, rituximab, rolapitant hydrochloride, romidepsin, romiplostim, rucaparib camsylate, ruxolitinib phosphate, selinexor, siltuximab, sipuleucel-t, sonidegib, sorafenib tosylate, sunitinib malate, tagraxofusp-erzs, talazoparib tosylate, talc, talimogene laherparepvec, tamoxifen citrate, temozolomide, temsirolimus, thalidomide, thioguanine, thiotepa, tisagenlecleucel, tocilizumab, topotecan hydrochloride, toremifene, trabectedin, trametinib, trastuzumab uridine, triacetate, valrubicin, vandetanib, vemurafenib, venetoclax, vincristine sulfate, vinorelbine tartrate, vismodegib, vorinostat, zanubrutinib, ziv-aflibercept, zoledronic acid, a pharmaceutically acceptable salt of any of these, or any combination thereof.

Embodiment 52. The method of embodiment 1, wherein the subject is a pediatric subject.

Embodiment 53. A method of treating a subject having a cancer, the method comprising: administering a therapeutically effective amount of:
i) an allogeneic cell,
ii) an isolated and purified HLA or a portion thereof, or
iii) a combination of (i) and (ii) to the subject,
wherein an antigen marker profile of a cell of the subject comprises at least one mismatched antigen marker as compared to an antigen marker profile of the allogeneic cell or the isolated and purified HLA or the portion thereof administered to the subject.

Embodiment 54. The method of embodiment 53, wherein the method further comprises administering an immunotherapy, and wherein the immunotherapy comprises a checkpoint inhibitor.

### EXAMPLES

### Example 1: Allo-Immunotherapy (AIM) Treatment of Prostate Cancer in Murine Model

The murine RM-1 prostate cancer model was used to evaluate an allo-immunotherapy (AIM) treatment with and without a checkpoint inhibitor. 500,000 RM1 cells were inoculated into the flank of C57BL/6 male mice. Allogeneic cells were sourced from subcutaneous fat of female Balb/c mice. At Day 0, tumor-bearing mice were randomized into treatment groups based on tumor volume. At Day 1, 0.4 cc of the allogeneic cells were implanted subcutaneously (0.2 cc on the left side and 0.2 cc on the right side) in proximity to the tumor (i.e., AIM (Allo-immunotherapy) alone group and AIM+anti-PD-1 group. Further, 10 mg/kg anti-PD-1 (RM1-14) in PBS was administered biweekly by intraperitoneal administration starting at day one to the anti-PD-1 group and the AIM+anti-PD-1 group. For the control, PBS was administered biweekly starting at day one by intraperitoneal administration to the vehicle group.

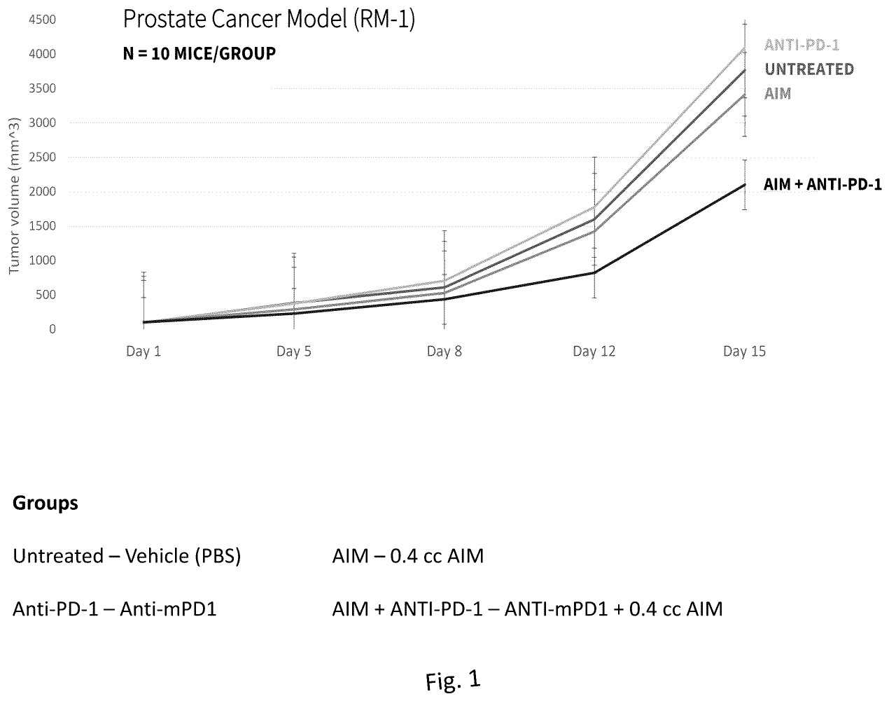

FIG. 1 shows the mean tumor volume on the Y-axis of a murine RM-1 prostate cancer model after being treated with: AIM (allo-immunotherapy), Anti-PD-1, AIM (allo-immunotherapy) and Anti-PD-1, and a untreated vehicle (PBS control). The X-axis shows the number of study days. The tumor volume was about 2100 mm³ in the AIM and Anti-PD-1 treatment group as compared to about 3500 mm³ to about 3890 mm³ for the other three treatment groups. A tumor volume of 2500 mm³ was measured in the AIM and Anti-PD-1 treatment group and 400 mm³ was subtracted from the total tumor volume as the AIM treatment dose (400 mm³) was indistinguishable from the tumor once the tumor was removed, similar adjustments were made to the AIM only treatment group. As shown in FIG. 1, there was a decrease in tumor growth in the RM-1 prostate cancer model when the allo-immunotherapy treatment was combined with an anti-PD-1 treatment, but not with anti-PD-1 alone. This data shows there was a synergistic treatment effect when the allo-immunotherapy treatment was combined with a checkpoint inhibitor treatment.

### Example 2: Treatment for Solid Tumor

A subject is diagnosed with a solid tumor by a diagnostic test. The subject receives a recommendation from a medical professional, based on a result of the diagnostic test, to receive a combination therapy. The combination therapy includes administration to the subject of an allogeneic cell and a cancer therapy.

### Example 3: Treatment for Prostate Cancer

A subject suffering from a prostate cancer receives consecutive administration of an allogeneic cell followed by a CTLA-4 checkpoint inhibitor to treat the prostate cancer. Administration includes 2 doses that occur 1× weekly.

### Example 4: Recombinant HLA Treatment

A subject suffering from a cancer receives a recombinant human leukocyte antigen (HLA) that is mismatched as compared to an antigen marker profile of a cell obtained from the subject to treat the cancer. The HLA therapy is administered by an injection.

### Example 5: Treatment for Bladder Cancer

A subject suffering from a bladder cancer receives by intraperitoneal injection administration an allogeneic cell followed by a PD-1 inhibitor to treat the bladder cancer. Administration of the allogeneic cell is provided at least 1 week prior to the PD-1 inhibitor.

### Example 6: Treatment for Breast Cancer

A subject suffering from a breast cancer receives by intraperitoneal injection administration of an allogeneic cell followed by a CAR T-cell therapy to treat the breast cancer. Administration of the allogeneic cell is provided at least 1 week prior to the CAR T cell therapy.

### Example 7: Treatment for Pancreatic Cancer

A subject suffering from a pancreatic cancer receives an allogeneic cell and a PD-1 checkpoint inhibitor concurrently to treat the pancreatic cancer. Efficacy of the PD-1 checkpoint inhibitor to treat the cancer is enhanced by at least about 70% when administered to the subject with the allogeneic cell as compared to administration of the PD-1 checkpoint inhibitor alone.

### Example 8: Clinical Trial

A plurality of subjects suffering from a cancer is evaluated in a clinical trial. The subjects are divided into two groups. One group of subjects is administered an allogeneic cell and a PD-1 inhibitor. A second group of subjects is administered the PD-1 inhibitor. Following administration, efficacy of the treatments for treating the cancer is evaluated.

### Example 9: Short-Term (8-Days) and Long-Term (60-Days) Tolerability Study

### Evaluating Immune Influx of AIM During the Short Term (8 Day) Tolerability Study

The tolerability of a MHC-mismatched subcutaneous allogeneic implant was evaluated. Six non-tumor bearing male C57BL/6 mice were anesthetized and implanted with 0.400 cc of AIM (allo-immunotherapy), sourced from BALB/c subcutaneous. The MHC haplotypes for the BALB/c mice was an MHC Class II allele of H2-Iad and a MHC Class I of H2-Kd. The MHC haplotypes for the C57BL/6 mice was an MHC Class II allele of H2-Iab and a MHC Class I of H2-Kb. The implanted AIM contained MHC mismatched subcutaneous fat. The dose was selected based on previous work suggesting a 0.860 cc+/−0.60 cc (range 0.1 cc-1.5 cc) as the average amount used for fat transfer and reconstruction research. Animals were monitored daily for body weight and clinical signs of health. Three animals were sacrificed on day 3 for immunohistochemistry, with the remaining animals sacrificed for immunohistochemistry on day 8. Six of six AIM recipient mice maintained body weight during in-life (t-test, alpha 0.05, to detect 10% BW change, data not shown), and major organs appeared normal upon visual necropsy (data not shown), suggesting that the AIM procedure was well tolerated. The non-tumor bearing pilot study suggested AIM therapy is likely tolerable beyond day 8.

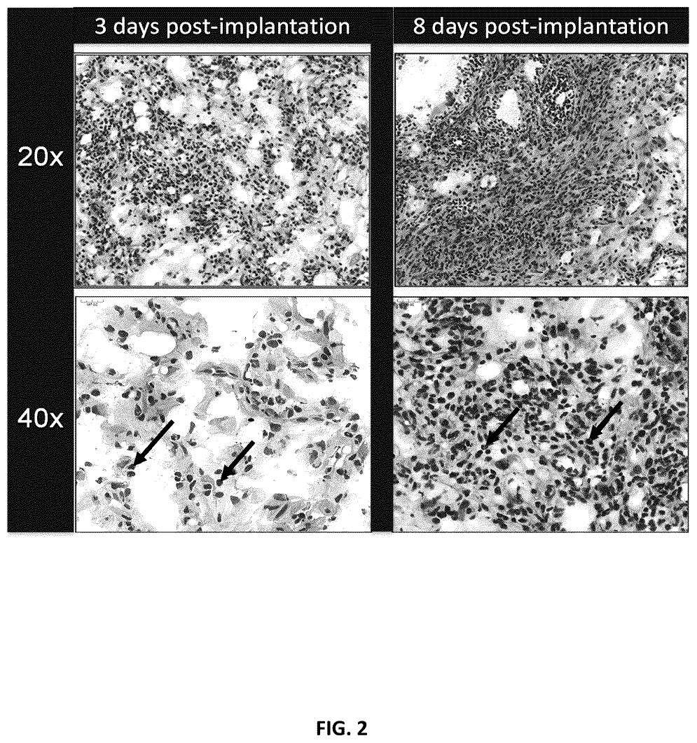

The lipid marker oil red confirmed that AIM is comprised of adipocytes prior to implantation (data not shown). Three animals were sacrificed on day 3, and another 3 on day 8 to prepare samples as formalin-fixed paraffin-embedded sections for hematoxylin and eosin stain (H&E). H&E showed a trend of increased influx of immune cells from the myeloid lineage over time, when comparing day 3 and day 8 samples as shown in FIG. 2. There was noted macrophage, polymorphonuclear cell (granulocyte), and fibrocyte infiltrates, suggesting AIM implantation induced an acute innate influx of immune cells, similar to that observed in acute transplant rejection.

### Evaluating Immune Influx of AIM During the Long Term (60 Day) Tolerability Study

Seven male C57BL/6 non-tumor bearing mice were anesthetized and implanted with 0.400 cc of AIM, sourced from BALB/c subcutaneous fat. Similar to the short-term tolerability study, all 7 animals treated with AIM maintained body weight and health throughout the 60-day study.

### Method for Subcutaneous Fat Harvesting and Implantation

Donor mice (BALB/c) were euthanized. Adipose tissue exposed from the skin was collected as abdominal subcutaneous fat (SCF). The SCF was collected by blunt dissecting and cutting it from the skin using sterile surgical scissors. In some cases, thoracic subcutaneous fat may also be collected. The SCF was implanted using the pocket method. The pocket method comprised creating a vertical incision then a pocket in the subcutaneous region of the skin using surgical scissors flanking the tumor. The fat was transferred using forceps or a syringe. The first half of the fat (0.200 cc) was transferred using forceps on one side of the tumor and the second half of the fat (0.200 cc) was transferred on the opposite side of the tumor. The incision was sealed. Hydryogel was provided ad lib during the recipient animals recovery phase.

### Example 10: Treatment for Prostate Cancer

A subject suffering from a prostate cancer is treated with a xenogeneic cell treatment followed by a CTLA-4 checkpoint inhibitor to treat the prostate cancer. Administration includes 1 dose of the therapies that occur 1× weekly.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define a scope of the disclosure and that methods and structures within the scope of these claims and their equivalents be covered thereby.

1. A method of treating a subject having a cancer, the method comprising:
administering a therapeutically effective amount of an allogeneic cell or a xenogeneic cell to the subject, wherein an antigen marker profile of a cell of the subject comprises at least one mismatched antigen marker as compared to an antigen marker profile of the allogeneic cell or the xenogeneic cell administered to the subject.

2. The method of claim 1, wherein the cancer comprises a malignant tumor.

3. The method of claim 1, wherein the antigen marker profile comprises a human leukocyte antigen (HLA) or a portion thereof.

4. The method of claim 1, wherein the at least one mismatched antigen marker is 2 mismatched antigen markers.

5. The method of claim 1, wherein the at least one mismatched antigen marker is 3 mismatched antigen markers.

6. The method of claim 1, wherein a cell transplant or tissue transplant comprising the allogeneic cell or the xenogeneic cell is administered to the subject.

7. The method of claim 1, further comprising: administering an isolated and purified HLA or a portion thereof to the subject, wherein the isolated and purified HLA or the portion thereof is mismatched to an antigen marker profile of a cell of the subject.

8. The method of claim 7, wherein the HLA or portion thereof is a recombinant HLA or recombinant portion thereof.

9. The method of claim 1, further comprising, administering a cancer therapy to the subject.

10. The method of claim 9, wherein the allogeneic cell or the xenogeneic cell and the cancer therapy are administered concurrently.

11. The method of claim 9, wherein the allogeneic cell or the xenogeneic cell and the cancer therapy are administered consecutively.

12. (canceled)

13. The method of claim 9, wherein the cancer therapy comprises a chimeric antigen receptor (CAR) T cell therapy, an immunotherapy, a chemotherapy, a radiation therapy, a surgery, a hormone therapy, a bone marrow transplant, a small molecule, a biologic, or any combination thereof.

14.-19. (canceled)

20. The method of claim 1, wherein the cancer comprises: a glioblastoma, a pancreatic cancer, a breast cancer, a prostate cancer, an ovarian cancer, a melanoma, a bladder cancer, a head or neck cancer, a kidney cancer, a liver cancer, a non-small cell lung cancer, or any combination thereof.

21.-24. (canceled)

25. The method of claim 1, wherein the administering of the allogeneic cell or the xenogeneic cell is by parenchymal injection, intra-thecal injection, intra-ventricular injection, intra-cisternal injection, intratumoral injection, subcutaneous injection, intraperitoneal injection, a surgical route, or any combination thereof.

26.-30. (canceled)

31. The method of claim 1, wherein the allogeneic cell or the xenogeneic cell comprises: a CAR T cell, a B cell, a T cell, an NK cell, a macrophage, a dendritic cell, an endothelial cell, a stem cell, an immune cell, a bone cell, a fat cell, a nerve cell, a skin cell, a pancreatic cell, a sex cell, a blood cell, a muscle cell, or an adipose cell.

32.-38. (canceled)

39. The method of claim 1, wherein upon the administering the therapeutically effective amount of the allogeneic cell or the xenogeneic cell to the subject, there is an increase in the number of immune cells to the site of the administering as measured by immunohistochemistry (IHC).

40.-85. (canceled)

86. The method of claim 1, wherein the subject has a PD-1 inhibitor or a PD-L1 inhibitor resistant tumor.

87. A cell bank comprising the allogeneic cell or the xenogeneic cell of claim 1.

88. A method of treating a subject having a cancer, the method comprising:
administering a therapeutically effective amount of a population of xenogeneic cells to the subject, wherein an antigen marker profile of a cell of the subject comprises at least one mismatched antigen marker as compared to an antigen marker profile of the population of xenogeneic cells administered to the subject, wherein the population of xenogenic cells comprise a population of adipose cells.

89. A method of treating a subject having a cancer, wherein the subject is a subject in need thereof, the method comprising: administering to the subject a therapeutically effective amount of:
i) an allogeneic cell or a xenogeneic cell;
ii) an isolated and purified HLA or a portion thereof, or
iii) a combination of (i) and (ii) to the subject in need thereof, thereby treating the subject having the cancer,
wherein an antigen marker profile of a cell of the subject comprises at least one mismatched antigen marker as compared to an antigen marker profile of the allogeneic cell, the xenogeneic cell, the isolated and purified HLA or the portion thereof, or any combination thereof, administered to the subject.
